(19) United States
(12) Patent Application Publication
(10) Pub. No.: US 2026/0345271 A1
(43) Pub. Date: Nov. 5, 2026

(54) MEDICAL INSTRUMENT DRIVE ASSEMBLY AND DOCKING SYSTEM

(71) Applicant: AURIS HEALTH, INC., Santa Clara, CA (US)

(72) Inventors: Adrian Tyler HAIRRELL, San Francisco, CA (US); Kathryn Mary PASWATERS, San Francisco, CA (US); Casey Teal LANDEY, San Francisco, CA (US); Akira Bryan UEDA, San Francisco, CA (US); Jeffrey WEINTRAUB, San Francisco, CA (US); Kenneth CHOU, Oakland, CA (US); Michael LO, San Francisco, CA (US); Nicolas DIBENEDETTO, San Francisco, CA (US); William CARTER-GIANNINI, San Jose, CA (US)

(73) Assignee: AURIS HEALTH, INC., Santa Clara, CA (US)

(21) Appl. No.: 19/733,752

(22) Filed: Jul. 7, 2026

Related U.S. Application Data
(62) Division of application No. 18/073,254, filed on Dec. 1, 2022.
Application No. 18/073,254 is a continuation of application No. PCT/IB2022/051380, filed on Feb. 16, 2022.
(60) Provisional application No. 63/150,418, filed on Feb. 17, 2021.

(51) Int. Cl. A61B 34/37; A61B 34/00; A61B 34/30

(52) U.S. Cl. CPC A61B 34/37; A61B 34/74; A61B 2034/301; A61B 2034/304; A61B 2034/305; A61B 2034/742

(57) ABSTRACT

A medical instrument drive assembly includes a base portion and one or more drive outputs rotatably coupled to the base portion, at least one of the one or more drive outputs including a first drive interface configured to mate with and drive a first type of medical instrument, and a second drive interface configured to mate with and drive a second type of medical instrument.

### RELATED APPLICATIONS

This application is a divisional of U.S. Patent Application No. 18/073,254, filed Dec. 1, 2022, entitled MEDICAL INSTRUMENT DRIVE ASSEMBLY AND DOCKING SYSTEM, which is a continuation of International Patent Application No. PCT/IB2022/051380, filed Feb. 16, 2022, entitled MEDICAL INSTRUMENT DRIVE ASSEMBLY AND DOCKING SYSTEM, which claims priority to U.S. Provisional Application No. 63/150,418, filed Feb. 17, 2021, entitled MEDICAL INSTRUMENT DRIVE ASSEMBLY AND DOCKING SYSTEM, the disclosure of which is hereby incorporated by reference in its entirety.

### BACKGROUND

Certain robotic medical procedures can involve interaction between one or more medical instruments and one or more medical instrument drive assemblies, which can include sterile adapters, robotic arm assemblies, and/or similar devices. In some cases, physicians may attach and/or mate a medical instrument to a medical instrument drive assembly while the physician navigates the medical instrument through a patient’s body.

### BRIEF DESCRIPTION OF THE DRAWINGS

Various embodiments are depicted in the accompanying drawings for illustrative purposes and should in no way be interpreted as limiting the scope of the inventions. In addition, various features of different disclosed embodiments can be combined to form additional embodiments, which are part of this disclosure. Throughout the drawings, reference numbers may be reused to indicate correspondence between reference elements.

FIG. 1 illustrates an embodiment of a robotic medical system including a medical instrument coupled to a robotic end effector in accordance with one or more embodiments.

FIG. 2 illustrates a robotic system arranged for diagnostic and/or therapeutic bronchoscopy in accordance with one or more embodiments.

FIG. 3 illustrates a table-based robotic system in accordance with one or more embodiments.

FIGS. 4-1 and 4-2 illustrate medical system components that may be implemented in any of the medical systems of FIGS. 1–3 in accordance with one or more embodiments.

FIG. 5 shows an exploded view of an instrument device manipulator assembly associated with a robotic arm configured to drive at least a medical instrument in accordance with one or more embodiments.

FIGS. 6A and 6B illustrate at least a portion of a drive assembly, which may include one or more adapters, having features configured to facilitate docking of one or more medical instruments in accordance with one or more embodiments.

FIGS. 7A and 7B provide illustrations of example platforms for use with one or more drive assemblies in accordance with one or more embodiments.

FIGS. 8A-8C illustrate a drive assembly including an adapter configured to drive one or more medical instruments, which can include a first medical instrument and/or a second medical instrument in accordance with one or more embodiments.

FIGS. 9A and 9B illustrate at least a portion of a drive assembly including an adapter comprising one or more magnetic elements and/or other alignment features configured to guide and/or attached to one or more medical instruments in accordance with one or more embodiments.

FIG. 10 provides a flow diagram for a process for driving one or more medical instruments at a drive assembly which may comprise one or more adapters (e.g., sterile adapters) for transferring force from a robotic system in accordance with one or more embodiments.

FIG. 11 illustrates at least a portion of a drive assembly including an adapter configured to drive multiple types of medical instruments in accordance with one or more embodiments, in accordance with one or more embodiments.

FIGS. 12A-12C illustrate a drive output configured to drive multiple types of medical instruments, in accordance with one or more embodiments.

FIG. 13 provides a flow diagram for a process for driving one or more medical instruments at a drive assembly which may comprise one or more adapters (e.g., sterile adapters) for transferring force from a robotic system in accordance with one or more embodiments.

### DETAILED DESCRIPTION

The headings provided herein are for convenience only and do not necessarily affect the scope or meaning of the claimed invention. Although certain preferred embodiments and examples are disclosed below, inventive subject matter extends beyond the specifically disclosed embodiments to other alternative embodiments and/or uses and to modifications and equivalents thereof. Thus, the scope of the claims that may arise herefrom is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

Although certain spatially relative terms, such as “outer,” “inner,” “upper,” “lower,” “below,” “above,” “vertical,” “horizontal,” “top,” “bottom,” “lateral,” and similar terms, are used herein to describe a spatial relationship of one device/element or anatomical structure to another device/element or anatomical structure, it is understood that these terms are used herein for ease of description to describe the positional relationship between element(s)/structures(s), such as with respect to the illustrated orientations of the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa. It should be understood that spatially relative terms, including those listed above, may be understood relative to a respective illustrated orientation of a referenced figure.

Certain reference numbers are re-used across different figures of the figure set of the present disclosure as a matter of convenience for devices, components, systems, features, and/or modules having features that may be similar in one or more respects. However, with respect to any of the embodiments disclosed herein, re-use of common reference numbers in the drawings does not necessarily indicate that such features, devices, components, or modules are identical or similar. Rather, one having ordinary skill in the art may be informed by context with respect to the degree to which usage of common reference numbers can imply similarity between referenced subject matter. Use of a particular reference number in the context of the description of a particular figure can be understood to relate to the identified device, component, aspect, feature, module, or system in that particular figure, and not necessarily to any devices, components, aspects, features, modules, or systems identified by the same reference number in another figure. Furthermore, aspects of separate figures identified with common reference numbers can be interpreted to share characteristics or to be entirely independent of one another. In some contexts features associated with separate figures that are identified by common reference numbers are not related and/or similar with respect to at least certain aspects.

The present disclosure provides systems, devices, and methods for facilitate interaction between one or more medical instruments and one or more medical instrument drive assemblies. With respect to medical instruments described in the present disclosure, the term “instrument” is used according to its broad and ordinary meaning and may refer to any type of tool, device, assembly, system, subsystem, apparatus, component, or the like. In some contexts herein, the term “device” may be used substantially interchangeably with the term “instrument.” Furthermore, the term “assembly” is used herein according to its broad and ordinary meaning and may refer to any device and/or set of devices. The term “drive assembly” is used herein according to its broad and ordinary meaning and may refer to any device and/or set of devices associated with driving one or more medical instruments.

### Medical Procedures

Although certain aspects of the present disclosure are described in detail herein in the context of renal, urological, and/or nephrological procedures, such as kidney stone removal/treatment procedures, it should be understood that such context is provided for convenience and clarity, and robotic and/or manual drive concepts disclosed herein are applicable to any suitable medical procedures, such as robotic bronchoscopy. However, as mentioned, description of the renal/urinary anatomy and associated medical issues and procedures is presented below to aid in the description of the inventive concepts disclosed herein.

In certain medical procedures, such as ureteroscopy procedures, elongate medical instruments that access the treatment site through an access sheath may be utilized to remove debris, such as kidney stones and stone fragments or other refuse or contaminant(s), from the treatment site. Kidney stone disease, also known as urolithiasis, is a medical condition that involves the formation in the urinary tract of a solid piece of material, referred to as “kidney stones,” “urinary stones,” “renal calculi,” “renal lithiasis,” or “nephrolithiasis.” Urinary stones may be formed and/or found in the kidneys, the ureters, and the bladder (referred to as “bladder stones”). Such urinary stones can form as a result of mineral concentration in urinary fluid and can cause significant abdominal pain once such stones reach a size sufficient to impede urine flow through the ureter or urethra. Urinary stones may be formed from calcium, magnesium, ammonia, uric acid, cystine, and/or other compounds or combinations thereof.

Several methods can be used for treating patients with kidney stones, including observation, medical treatments (such as expulsion therapy), non-invasive treatments (such as extracorporeal shock wave lithotripsy (ESWL)), minimally invasive or surgical treatments (such as ureteroscopy and percutaneous nephrolithotomy (“PCNL”)), and so on. In some approaches (e.g., ureteroscopy and PCNL), the physician gains access to the stone, the stone is broken into smaller pieces or fragments, and the relatively small stone fragments/particulates are extracted from the kidney using a basketing device and/or aspiration.

In some procedures, surgeons may insert an endoscope (e.g., ureteroscope) into the urinary tract through the urethra to remove urinary stones from the bladder and ureter. Typically, a ureteroscope includes a camera at its distal end configured to enable visualization of the urinary tract. The ureteroscope can also include, or allow for placement in a working channel of the ureteroscope, a lithotripsy device configured to capture or break apart urinary stones. During a ureteroscopy procedure, one physician/technician may control the position of the ureteroscope, while another physician/technician may control the lithotripsy device(s).

In some procedures, such as procedures for removing relatively large stones/fragments, physicians may use a percutaneous nephrolithotomy (“PCNL”) technique that involves inserting a nephroscope through the skin (i.e., percutaneously) and intervening tissue to provide access to the treatment site for breaking-up and/or removing the stone(s). A percutaneous-access device (e.g., nephroscope, sheath, sheath assembly, and/or catheter) used to provide an access channel to the target anatomical site (and/or a direct-entry endoscope) may include one or more fluid channels for providing irrigation fluid flow to the target site and/or aspirating fluid from the target site (e.g., through passive outflow and/or active suction).

For ureteroscopic procedures, a physician may implement a procedure to break a relatively large kidney stone into a relatively smaller fragments to facilitate extraction thereof. For example, certain instruments may be utilized to break the stone into smaller fragments, such as by lasing, or through other application of cleaving force to the kidney stone. According to some procedures, a basketing device/system may be used to capture the relatively smaller stone fragment(s) and extract them from the treatment site out of the patient. Generally, when a stone is captured, the surgeon may wish to quickly extract the stone through the uretereral access sheath prior to opening the basket to deposit/drop the stone into a specimen collection structure or area, after which the basket may be closed and reinserted (e.g., within a working channel of an endoscope/ureteroscope) through the access sheath for the purpose of extracting remaining stones or stone fragments, should there be any.

Robotic-assisted ureteroscopic procedures can be implemented in connection with various medical procedures, such as kidney stone removal procedures, wherein robotic tools can enable a physician/urologist to perform endoscopic target access as well as percutaneous access/treatment. Advantageously, aspects of the present disclosure relate to systems, devices, and methods for managing and/or facilitating interaction (which can including attaching, mating, contacting, locking, latching, removing, guiding, driving, among other interactions described herein) between one or more medical instruments (which can include endoscopes/ureteroscopes, basketing devices/systems, lithotripsy device(s), and/or various other instruments) and/or medical instrument drive assemblies (which can include adapters, sterile adapters, robotic arm devices, and/or various other devices).

### Medical System

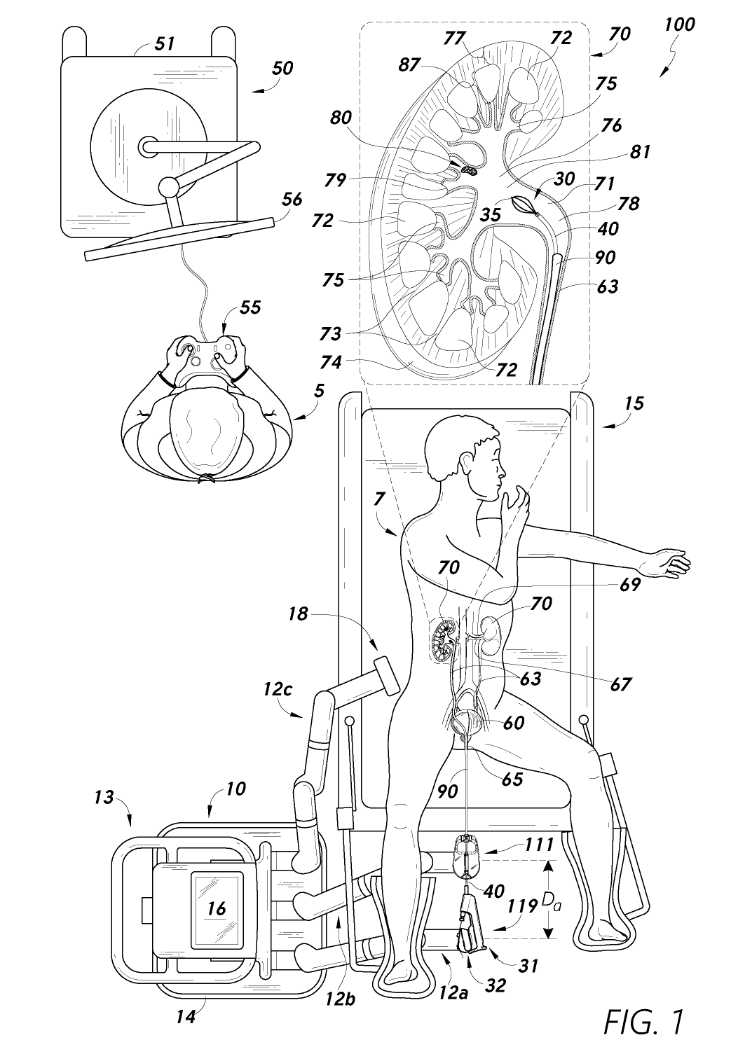

FIG. 1 illustrates an example medical system 100 for performing various medical procedures in accordance with aspects of the present disclosure. The medical system 100 may be used for, for example, endoscopic (e.g., ureteroscopic) procedures. As referenced and described above, certain ureteroscopic procedures involve the treatment/removal of kidney stones. In some implementations, kidney stone treatment can benefit from the assistance of certain robotic technologies/devices. Robotic medical solutions can provide relatively higher precision, superior control, and/or superior hand-eye coordination with respect to certain instruments compared to strictly manual procedures. For example, robotic-assisted ureteroscopic access to the kidney in accordance with some procedures can advantageously enable a urologist to individually perform both endoscope control and basketing control.

Although the system 100 of FIG. 1 is presented in the context of a ureteroscopic procedure, it should be understood that the principles disclosed herein may be implemented in any type of endoscopic procedure. Furthermore, several of the examples described herein relate to object removal procedures involving the removal of kidney stones from a kidney. The present disclosure, however, is not limited only to kidney stone removal. For example, the following description is also applicable to other surgical or medical operations or medical procedures concerned with the removal of objects from a patient, including any object that can be removed from a treatment site or patient cavity (e.g., the esophagus, ureter, intestine, eye, etc.) via percutaneous and/or endoscopic access, such as, for example, gallbladder stone removal, lung (pulmonary/transthoracic) tumor biopsy, or cataract removal.

The medical system 100 includes a robotic system 10 (e.g., mobile robotic cart) configured to engage with and/or control a medical instrument 19 (e.g., ureteroscope) including a proximal handle 31 and a shaft 40 coupled to the handle 31 at a proximal portion thereof to perform a direct-entry procedure on a patient 7. The term “direct-entry” is used herein according to its broad and ordinary meaning and may refer to any entry of instrumentation through a natural or artificial opening in a patient’s body. For example, with reference to FIG. 1, the direct entry of the scope/shaft 40 into the urinary tract of the patient 7 may be made via the urethra 65. The term “shaft” is used herein according to its broad and ordinary meaning and may refer to any type of elongate cylinder, tube, scope (e.g., endoscope), prism (e.g., rectangular, oval, elliptical, or oblong prism), wire, or similar, regardless of cross-sectional shape. It should be understood that any reference herein to a “shaft” or “instrument shaft” can be understood to possibly refer to an endoscope.

It should be understood that the direct-entry instrument 19 may be any type of shaft-based medical instrument, including an endoscope (such as a ureteroscope), catheter (such as a steerable or non-steerable catheter), nephroscope, laparoscope, or other type of medical instrument. Embodiments of the present disclosure relating to ureteroscopic procedures for removal of kidney stones through a ureteral access sheath (e.g., the ureteral access sheath 90) are also applicable to solutions for removal of objects through percutaneous access, such as through a percutaneous access sheath. For example, instrument(s) may access the kidney percutaneously through, for example, a percutaneous access sheath to capture and remove kidney stones. The term “percutaneous access” is used herein according to its broad and ordinary meaning and may refer to entry, such as by puncture and/or minor incision, of instrumentation through the skin of a patient and any other body layers necessary to reach a target anatomical location associated with a procedure (e.g., the calyx network of the kidney 70).

The medical system 100 includes a control system 50 configured to interface with the robotic system 10, provide information regarding the procedure, and/or perform a variety of other operations. For example, the control system 50 can include various input/output components 258 which can include one or more display(s) 56 configured to present certain information to assist the physician 5 and/or other technician(s) or individual(s). The medical system 100 can include a table 15 configured to hold the patient 7. The system 100 may further include an electromagnetic (EM) field generator 18, which may be held by one or more of the robotic arms 12 of the robotic system 10 or may be a stand-alone device. Although the various robotic arms 12 are shown in various positions and coupled to various tools/devices, it should be understood that such configurations are shown for convenience and illustration purposes, and such robotic arms may have different configurations over time and/or at different points during a medical procedure. Furthermore, the robotic arms 12 may be coupled to different devices/instruments than shown in FIG. 1, and in some cases or periods of time, one or more of the arms may not be utilized or coupled to a medical instrument (e.g., instrument manipulator/coupling). Roll of the shaft 40 may be controlled robotically and/or manually, such as through operation of an end effector associated with the robot arm 12a, wherein such operation may be controlled by the control system 50 and/or robotic system 10. The term “end effector” is used herein according to its broad and ordinary meaning and may refer to any type of robotic manipulator device, component, and/or assembly. Where an adapter, such as a sterile adapter, is coupled to a robotic end effector or other robotic manipulator, the term “end effector” may refer to the adapter (e.g., sterile adapter), or any other robotic manipulator device, component, or assembly associated with and/or coupled to the end effector. In some contexts, the combination of a robotic end effector and adapter may be referred to as an instrument manipulator assembly, wherein such assembly may or may not also include a medical instrument (or instrument handle/base) physically coupled to the adapter and/or end effector. The terms “robotic manipulator” and “robotic manipulator assembly are used according to their broad and ordinary meanings, and may refer to a robotic end effector and/or sterile adapter or other adapter component coupled to the end effector, either collectively or individually. For example, “robotic manipulator” or “robotic manipulator assembly” may refer to an instrument device manipulator (IDM) including one or more drive outputs, whether embodied in a robotic end effector, sterile adapter, and/or other component(s).

In an example use case, if the patient 7 has a kidney stone (or stone fragment) 80 located in a kidney 70, the physician may execute a procedure to remove the stone 80 through the urinary tract (63, 60, 65). In some embodiments, the physician 5 can interact with the control system 50 and/or the robotic system 10 to cause/control the robotic system 10 to advance and navigate the medical instrument shaft 40 (e.g., a scope) from the urethra 65, through the bladder 60, up the ureter 63, and into the renal pelvis 71 and/or calyx network of the kidney 70 where the stone 80 is located. The physician 5 can further interact with the control system 50 and/or the robotic system 10 to cause/control the advancement of a basketing device 30 through a working channel of the instrument shaft 40, wherein the basketing device 30 is configured to facilitate capture and removal of a kidney stone or stone fragment. The control system 50 can provide information via the display(s) 56 that is associated with the medical instrument 40, such as real-time endoscopic images captured therewith, and/or other instruments of the system 100, to assist the physician 5 in navigating/controlling such instrumentation.

The renal anatomy is described herein for reference with respect to certain medical procedures relating to aspects of the present inventive concepts. The kidneys 70, shown roughly in typical anatomical position in FIG. 1, generally comprise two bean-shaped organs located on the left and right sides, respectively, in the retroperitoneal space. In adult humans, the kidneys are generally about 11 cm in height/length. The kidneys receive blood from the paired renal arteries 69; blood exits the kidney via the paired renal veins 67. Each kidney 70 is fluidly coupled with a respective ureter 63, which generally comprises a tube that carries excreted urine from the kidney 70 to the bladder 60.

The kidneys 70 are typically located relatively high in the abdominal cavity and lie in a retroperitoneal position at a slightly oblique angle. The asymmetry within the abdominal cavity, generally caused by the position of the liver, results in the right kidney (shown in detail in FIG. 1) typically being slightly lower and smaller than the left, and being placed slightly more to the middle than the left kidney. On top of each kidney is an adrenal gland (not shown). The upper parts of the kidneys 70 are partially protected by the 11^{th} and 12^{th} ribs (not shown). Each kidney, with its adrenal gland, is generally surrounded by two layers of fat: the perirenal fat present between renal fascia and renal capsule and pararenal fat superior to the renal fascia.

The kidneys 70 participate in the control of the volumes of various body fluid compartments, fluid osmolality, acid-base balance, various electrolyte concentrations, and removal of toxins. The kidneys 70 provide filtration functionality by secreting certain substances and reabsorbing others. Examples of substances secreted into the urine are hydrogen, ammonium, potassium and uric acid. In addition, the kidneys also carry out various other functions, such as hormone synthesis, and others.

A recessed area on the concave border of the kidney 70 is the renal hilum 81, where the renal artery 69 (not shown in the detailed view of the kidney 70) enters the kidney 70 and the renal vein 67 (not shown in detailed view) and ureter 63 leave. The kidney 70 is surrounded by tough fibrous tissue, the renal capsule 74, which is itself surrounded by perirenal fat, renal fascia, and pararenal fat. The anterior (front) surface of these tissues is the peritoneum, while the posterior (rear) surface is the transversalis fascia.

The functional substance, or parenchyma, of the kidney 70 is divided into two major structures: the outer renal cortex 77 and the inner renal medulla 87. These structures take the shape of a plurality of generally cone-shaped renal lobes, each containing renal cortex surrounding a portion of medulla called a renal pyramid 72. Between the renal pyramids 72 are projections of cortex called renal columns 73. Nephrons (not shown in detail in FIG. 1), the urine-producing functional structures of the kidney, span the cortex 77 and medulla 87. The initial filtering portion of a nephron is the renal corpuscle, which is located in the cortex and is followed by a renal tubule that passes from the cortex deep into the medullary pyramids. Part of the renal cortex, a medullary ray, is a collection of renal tubules that drain into a single collecting duct.

The tip/apex, or papilla 79, of each renal pyramid empties urine into a respective minor calyx 75; minor calyces 75 empty into major calyces 76, and major calyces 76 empty into the renal pelvis 71, which transitions to the ureter 63. The manifold-type collection of minor and major calyces may be referred to herein as the “calyx network” of the kidney. At the hilum 81, the ureter 63 and renal vein 67 exit the kidney and the renal artery 69 enters. Hilar fat and lymphatic tissue with lymph nodes surround these structures. The hilar fat is contiguous with a fat-filled cavity called the renal sinus. The renal sinus collectively contains the renal pelvis 71 and calyces 75, 76 and separates these structures from the renal medullary tissue. The funnel/tubular-shaped anatomy associated with the calyces can be referred to as the infundibulum/infundibula. That is, an infundibulum generally leads to the termination of a calyx where a papilla is exposed within the calyx.

With further reference to the medical system 100, the medical instrument shaft 40 (e.g., scope, directly-entry instrument, etc.) can be advanced into the kidney 70 through the urinary tract. Specifically, a ureteral access sheath 90 may be disposed within the urinary tract to an area near the kidney 70. The shaft 40 may be passed through the ureteral access sheath 90 to gain access to the internal anatomy of the kidney 70, as shown. Once at the site of the kidney stone 80 (e.g., within a target calyx 75 of the kidney 70 through which the stone 80 is accessible), the medical instrument 19 and/or shaft 40 thereof can be used to channel/direct the basketing device 30 to the target location. Once the stone 80 has been captured in the distal basket portion 35 of the basketing device/assembly 30, the utilized ureteral access path may be used to extract the kidney stone 80 from the patient 7.

The various scope/shaft-type instruments disclosed herein, such as the shaft 40 of the system 100, can be configured to navigate within the human anatomy, such as within a natural orifice or lumen of the human anatomy. The terms “scope” and “endoscope” are used herein according to their broad and ordinary meanings, and may refer to any type of elongate (e.g., shaft-type) medical instrument having image generating, viewing, and/or capturing functionality and being configured to be introduced into any type of organ, cavity, lumen, chamber, or space of a body. A scope can include, for example, a ureteroscope (e.g., for accessing the urinary tract), a laparoscope, a nephroscope (e.g., for accessing the kidneys), a bronchoscope (e.g., for accessing an airway, such as the bronchus), a colonoscope (e.g., for accessing the colon), an arthroscope (e.g., for accessing a joint), a cystoscope (e.g., for accessing the bladder), colonoscope (e.g., for accessing the colon and/or rectum), borescope, and so on. Scopes/endoscopes, in some instances, may comprise an at least partially rigid and/or flexible tube, and may be dimensioned to be passed within an outer sheath, catheter, introducer, or other lumen-type device, or may be used without such devices.

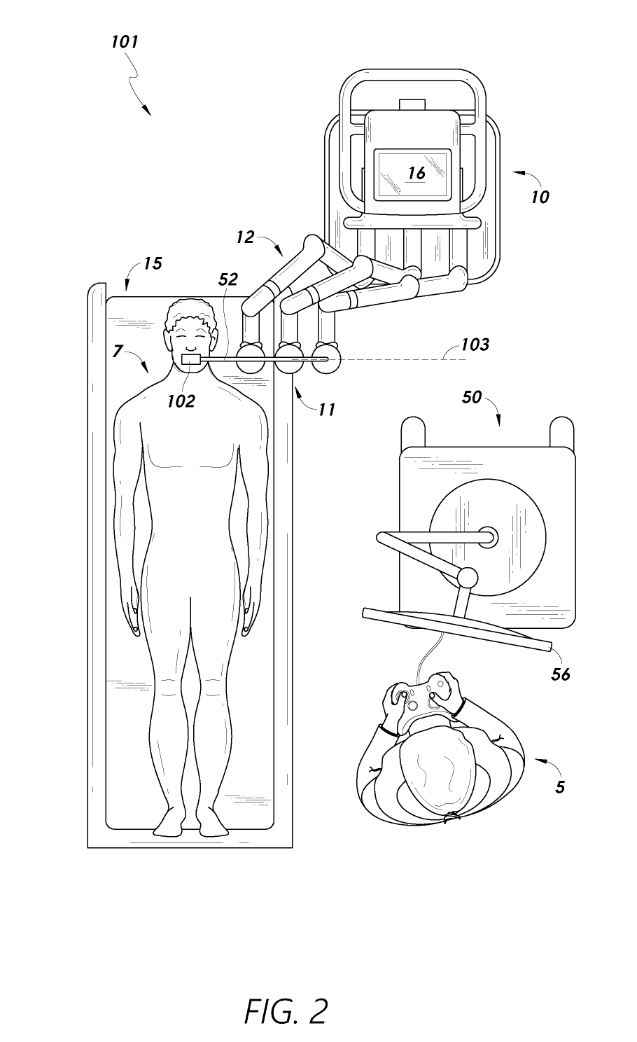

FIG. 2 illustrates a cart-based robotic system 101 arranged for diagnostic and/or therapeutic bronchoscopy in accordance with one or more embodiments. During a bronchoscopy, the arm(s) 12 of the robotic system 10 may be configured to drive a medical instrument shaft 52, such as a steerable endoscope, which may be a procedure-specific bronchoscope for bronchoscopy, through a natural orifice access point (e.g., the mouth of the patient 7 positioned on a table 15 in the present example) to deliver diagnostic and/or therapeutic tools. As shown, the robotic system 10 (e.g., cart) may be positioned proximate to the patient’s upper torso in order to provide access to the access point. Similarly, the robotic arms 12 may be actuated to position the bronchoscope/shaft 52 relative to the access point. The arrangement in FIG. 2 may also be utilized when performing a gastro-intestinal (GI) procedure with a gastroscope, a specialized endoscope for GI procedures.

Once the robotic system 10 is properly positioned, the robotic arms 12 may insert the steerable endoscope 52 into the patient robotically, manually, or a combination thereof. The steerable endoscope 52 may comprise at least two telescoping parts, such as an inner leader portion and an outer sheath portion, each portion coupled to a separate instrument feeder from the set of instrument feeders and/or instrument handles 11, each instrument feeder/handle being coupled to the distal end of a respective robotic arm 12. This linear arrangement of the feeder(s)/handle(s) 11 can create a “virtual rail” 103 that may be repositioned in space by manipulating the one or more robotic arms 12 into different angles and/or positions.

The endoscope 52 may be directed down the patient’s trachea and lungs after insertion using precise commands from the robotic system 10 until reaching the target operative site. For example, the endoscope 52 may be directed to deliver a biopsy needle to a target, such as, for example, a lesion or nodule within the lungs of a patient. The needle may be deployed down a working channel that runs the length of the endoscope to obtain a tissue sample to be analyzed by a pathologist. Depending on the pathology results, additional tools may be deployed down the working channel of the endoscope for additional biopsies. For example, when a nodule is identified as being malignant, the endoscope 52 may endoscopically deliver tools to resect the potentially cancerous tissue. In some instances, diagnostic and therapeutic treatments can be delivered in separate procedures. In those circumstances, the endoscope 52 may also be used to deliver a fiducial to “mark” the location of the target nodule as well. In other instances, diagnostic and therapeutic treatments may be delivered during the same procedure.

In the system 101, a patient introducer 102 is attached to the patient 7 via a port (not shown; e.g., surgical tube). The curvature of the introducer 102 may enable the robotic system 10 to manipulate the instrument 52 from a position that is not in direct axial alignment with the patient-access port, thereby allowing for greater flexibility in the placement of the robotic system 10 within the room. Further, the curvature of the introducer 102 may allow the robotic arms 12 of the robotic system 10 to be substantially horizontally aligned with the patient introducer 102, which may facilitate manual movement of the robotic arm(s) 12 if needed.

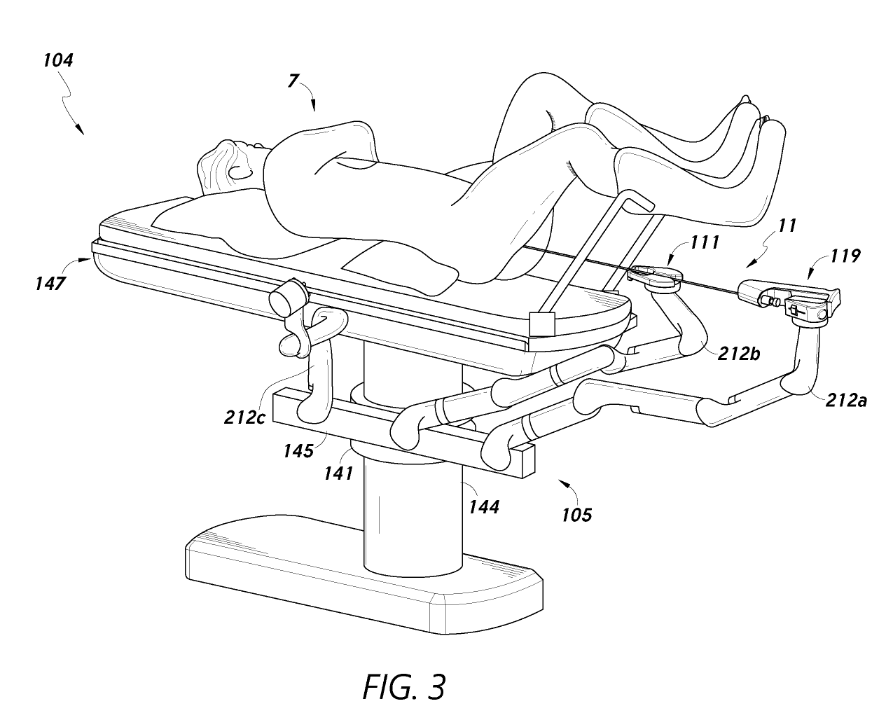

FIG. 3 illustrates a table-based robotic system 104 in accordance with one or more embodiments of the present disclosure. The system 104 incorporates robotic components 105 with a platform 147, thereby allowing for a reduced amount of capital equipment within the operating room compared to some cart-based robotic systems, which can allow greater access to the patient 7 in some instances. Much like in the cart-based systems, the instrument device manipulator assemblies associated with the robotic arms 212 of the system 104 may generally comprise instruments and/or instrument feeders that are designed to manipulate an elongated medical instrument/shaft, such as a catheter or the like, along a virtual rail or path.

As shown, the robotic-enabled table system 104 can include a column 144 coupled to one or more carriages 141 (e.g., ring-shaped movable structures), from which the one or more robotic arms 212 may emanate. The carriage(s) 141 may translate along a vertical column interface that runs at least a portion of the length of the column 144 to provide different vantage points from which the robotic arms 212 may be positioned to reach the patient 7. The carriage(s) 141 may rotate around the column 144 in some embodiments using a mechanical motor positioned within the column 144 to allow the robotic arms 212 to have access to multiples sides of the table 104. Rotation and/or translation of the carriage(s) 141 can allow the system 104 to align the medical instruments, such as endoscopes and catheters, into different access points on the patient. By providing vertical adjustment, the robotic arms 212 can advantageously be configured to be stowed compactly beneath the platform 147 of the table system 104 and subsequently raised during a procedure.

The robotic arms 212 may be mounted on the carriage(s) 141 through one or more arm mounts 145, which may comprise a series of joints that may individually rotate and/or telescopically extend to provide additional configurability to the robotic arms 212. The column 144 structurally provides support for the table platform 147 and a path for vertical translation of the carriage(s) 141. The column 144 may also convey power and control signals to the carriage(s) 141 and/or the robotic arms 212 mounted thereon. The system 104 can include certain control circuitry configured to control driving and/or roll of the instrument shaft using the instrument feeder 11, which may be coupled to an end effector of one of the arms 212, wherein the instrument feeder 11 is controlled to automatically modify axial driving speed with respect to the elongate instrument (e.g., endoscope) 119 based on a determined position of a distal end of the instrument 119. For example, when the distal end of the instrument 119 is positioned at a predetermined automatic pause location, the instrument feeder 11 can be controlled/driven to automatically pause/stop axial retraction to allow for specimen collection, as described in detail herein.

With reference to FIGS. 1–3 and 4-1, which shows an example embodiment of the control systems of any of FIGS. 1–3, the relevant control system 50 can be configured to provide various functionality to assist in performing a medical procedure. In some embodiments, the control system 50 can be coupled to the robotic system 10 and operate in cooperation therewith to perform a medical procedure on the patient 7. For example, the control system 50 can communicate with the robotic system 10 via a wireless or wired connection (e.g., to control the robotic system 10). Further, in some embodiments, the control system 50 can communicate with the robotic system 10 to receive position data therefrom relating to the position of the distal end of the scope 40, access sheath 90, or basketing device 30. Such positional data relating to the position of the scope 40, access sheath 90, or basketing device 30 may be derived using one or more electromagnetic sensors associated with the respective components, scope image processing functionality, and/or based at least in part on robotic system data (e.g., arm position data, known parameters/dimensions of the various system components, etc.). Moreover, in some embodiments, the control system 50 can communicate with the table 15 to position the table 15 in a particular orientation or otherwise control the table 15. In some embodiments, the control system 50 can communicate with the EM field generator 18 to control generation of an EM field in an area around the patient 7 and/or around the instrument feeder 11.

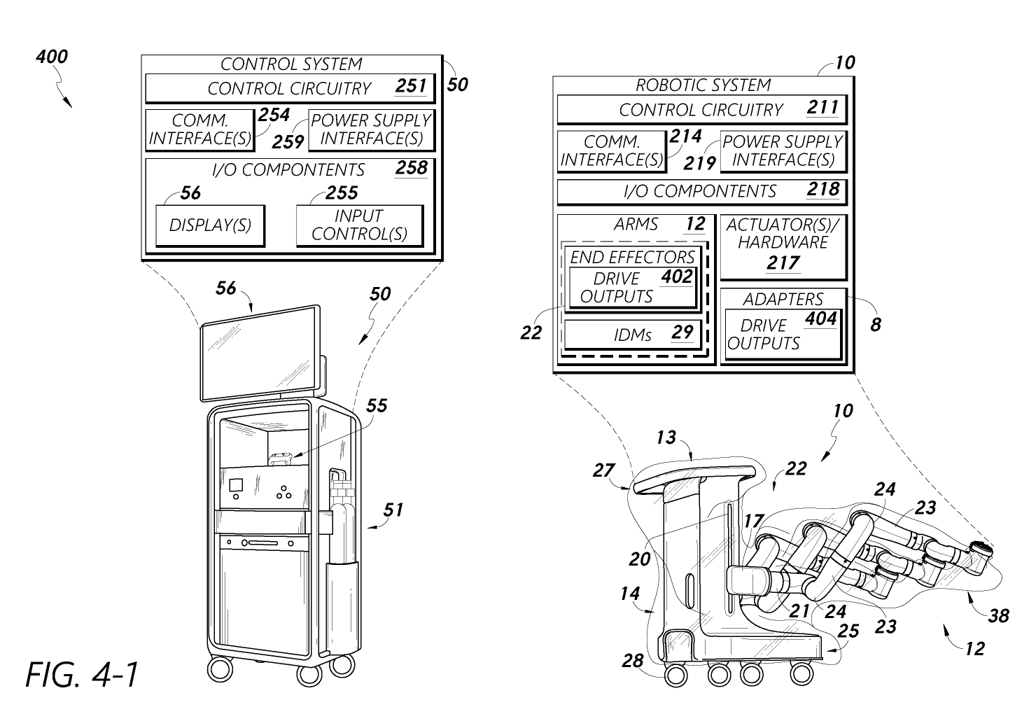

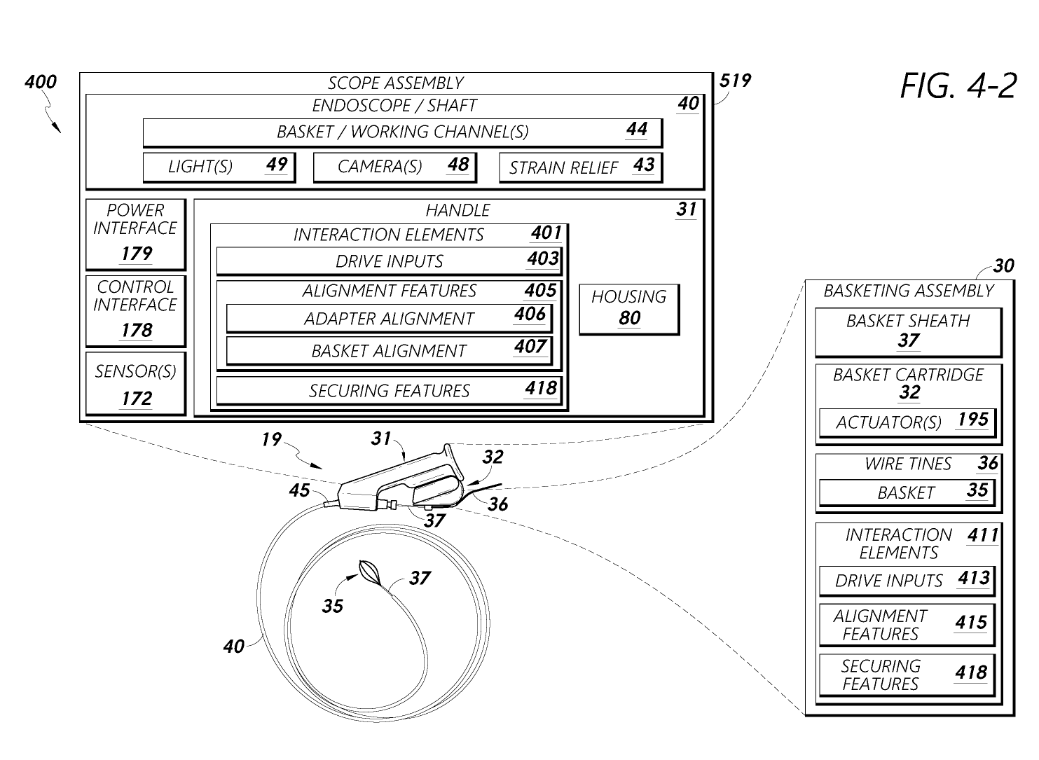

FIG. 4-1 further shows an example embodiment of the robotic systems 400 of any of FIGS. 1–3. The robotic system 10 can be configured to at least partly facilitate execution of a medical procedure. The robotic system 10 can be arranged in a variety of ways depending on the particular procedure. The robotic system 10 can include one or more robotic arms 12 configured to engage with and/or control, for example, the scope 40 and/or the basketing device/system 30 to perform one or more aspects of a procedure. As shown, each robotic arm 12 can include multiple arm segments 23 coupled to joints 24, which can provide multiple degrees of movement/freedom. In the example of FIG. 1, the robotic system 10 is positioned proximate to the patient’s legs and the robotic arms 12 are actuated to engage with and position the scope 40 for access into an access opening, such as the urethra 65 of the patient 7. When the robotic system 10 is properly positioned, the scope 40 can be inserted into the patient 7 robotically using the robotic arms 12, manually by the physician 5, or a combination thereof. With reference to FIG. 1, a scope-driver/feeder instrument coupling 11 (i.e., instrument device manipulator (IDM)) can be attached to the distal end effector 22 of one of the arms 12b to facilitate robotic control/advancement of the scope 40. Another 12a of the arms may have associated therewith an instrument coupling/manipulator 19 that is configured to facilitate advancement and operation of the basketing device 30. The instrument coupling 19 may further provide a handle 31 for the scope 40, wherein the scope 40 is physically coupled to the handle 31 at a proximal end of the scope 40. The scope 40 may include one or more working channels through which additional tools, such as lithotripters, basketing devices, forceps, etc., can be introduced into the treatment site.

The robotic system 10 can be coupled to any component of the medical system 100, such as to the control system 50, the table 15, the EM field generator 18, the scope 40, the basketing system 30, and/or any type of percutaneous-access instrument (e.g., needle, catheter, nephroscope, etc.). In some embodiments, the robotic system 10 is communicatively coupled to the control system 50. For example, the robotic system 10 may be configured to receive control signals from the control system 50 to perform certain operations, such as to position one or more of the robotic arms 12 in a particular manner, manipulate the scope 40, manipulate the basketing system 30, and so on. In response, the robotic system 10 can control, using certain control circuitry 211, actuators 217, and/or other components of the robotic system 10, a component of the robotic system 10 to perform the operations. In some embodiments, the robotic system 10 and/or control system 50 is/are configured to receive images and/or image data from the scope 40 representing internal anatomy of the patient 7 and/or portions of the access sheath or other device components.

The robotic system 10 generally includes an elongated support structure 14 (also referred to as a “column”), a robotic system base 25, and a console 13 at the top of the column 14. The column 14 may include one or more arm supports 17 (also referred to as a “carriage”) for supporting the deployment of the one or more robotic arms 12 (three shown in FIG. 1). The arm support 17 may include individually configurable arm mounts that rotate along a perpendicular axis to adjust the base of the robotic arms 12 for desired positioning relative to the patient.

The arm support 17 may be configured to vertically translate along the column 14. In some embodiments, the arm support 17 can be connected to the column 14 through slots 20 that are positioned on opposite sides of the column 14 to guide the vertical translation of the arm support 17. The slot 20 contains a vertical translation interface to position and hold the arm support 17 at various vertical heights relative to the robotic system base 25. Vertical translation of the arm support 17 allows the robotic system 10 to adjust the reach of the robotic arms 12 to meet a variety of table heights, patient sizes, and physician preferences. Similarly, the individually configurable arm mounts on the arm support 17 can allow the robotic arm base 21 of robotic arms 12 to be angled in a variety of configurations.

The robotic arms 12 may generally comprise robotic arm bases 21 and end effectors 22, separated by a series of linking arm segments 23 that are connected by a series of joints 24, each joint comprising one or more independent actuators 217. Each actuator may comprise an independently controllable motor. Each independently controllable joint 24 can provide or represent an independent degree of freedom available to the robotic arm. In some embodiments, each of the arms 12 has seven joints, and thus provides seven degrees of freedom, including “redundant” degrees of freedom. Redundant degrees of freedom allow the robotic arms 12 to position their respective end effectors 22 at a specific position, orientation, and trajectory in space using different linkage positions and joint angles. This allows for the system to position and direct a medical instrument from a desired point in space while allowing the physician to move the arm joints into a clinically advantageous position away from the patient to create greater access, while avoiding arm collisions.

The robotic system base 25 balances the weight of the column 14, arm support 17, and arms 12 over the floor. Accordingly, the robotic system base 25 and/or control system 50 may house certain relatively heavier components, such as electronics, motors, power supply interface(s) 219, 259, as well as components that selectively enable movement or immobilize the robotic system 10 and/or control system 50. For example, the robotic system base 25 can include wheel-shaped casters 28 that allow for the robotic system to easily move around the operating room prior to a procedure. After reaching the appropriate position, the casters 28 may be immobilized using wheel locks to hold the robotic system 10 in place during the procedure.

Positioned at the upper end of column 14, the console 13 can provide both a user interface for receiving user input and a display screen 16 (or a dual-purpose device such as, for example, a touchscreen) to provide the physician/user with both pre-operative and intra-operative data. Potential pre-operative data on the console/display 16 or display 56 may include pre-operative plans, navigation and mapping data derived from pre-operative computerized tomography (CT) scans, and/or notes from pre-operative patient interviews. Intra-operative data on display may include optical information provided from the tool, sensor and coordinate information from sensors, as well as vital patient statistics, such as respiration, heart rate, and/or pulse. The console 13 may be positioned and tilted to allow a physician to access the console from the side of the column 14 opposite arm support 17. From this position, the physician may view the console 13, robotic arms 12, and patient while operating the console 13 from behind the robotic system 10. As shown, the console 13 can also include a handle 27 to assist with maneuvering and stabilizing the robotic system 10.

The end effector 22 of each of the robotic arms 12 may comprise, or be configured to have coupled thereto, an instrument device manipulator (IDM) 29, which may be attached using a sterile adapter component in some instances. The combination of the end effector 22 and associated IDM, as well as any intervening mechanics or couplings (e.g., sterile adapter), can be referred to as a manipulator assembly. In some embodiments, the IDM 29 can be removed and replaced with a different type of IDM, for example, a first type 111 of IDM/instrument may be configured to manipulate an endoscope/shaft, while a second type 119 of IDM/instrument may be associated with the shaft (e.g., coupled to a proximal portion thereof) and configured to roll and/or articulate the shaft, and/or manipulate a basketing device. Another type of IDM/instrument may be configured to hold an electromagnetic field generator 18. An IDM can provide power 179 and control 178 interfaces. For example, the interfaces can include connectors to transfer pneumatic pressure, electrical power, electrical signals, and/or optical signals from the robotic arm 12 to the IDM. The IDMs 29 may be configured to manipulate medical instruments (e.g., surgical tools/instruments), such as the scope 40, using techniques including, for example, direct drives, harmonic drives, geared drives, belts and pulleys, magnetic drives, and the like. In some embodiments, the device manipulators 29 can be attached to respective ones of the robotic arms 12, wherein the robotic arms 12 are configured to insert or retract the respective coupled medical instruments into or out of the treatment site.

As referenced above, the system 100 can include certain control circuitry configured to perform certain of the functionality described herein, including the control circuitry 211 of the robotic system 10 and the control circuitry 251 of the control system 50. That is, the control circuitry of the systems 100, 101, 104 may be part of the robotic system 10, the control system 50, or some combination thereof. Therefore, any reference herein to control circuitry may refer to circuitry embodied in a robotic system, a control system, or any other component of a medical system, such as the medical systems 100, 101, and 104 shown in FIGS. 1–3, respectively. The term “control circuitry” is used herein according to its broad and ordinary meaning, and may refer to any collection of processors, processing circuitry, processing modules/units, chips, dies (e.g., semiconductor dies including one or more active and/or passive devices and/or connectivity circuitry), microprocessors, micro-controllers, digital signal processors, microcomputers, central processing units, field-programmable gate arrays, programmable logic devices, state machines (e.g., hardware state machines), logic circuitry, analog circuitry, digital circuitry, and/or any device that manipulates signals (analog and/or digital) based on hard coding of the circuitry and/or operational instructions. Control circuitry referenced herein may further include one or more circuit substrates (e.g., printed circuit boards), conductive traces and vias, and/or mounting pads, connectors, and/or components. Control circuitry referenced herein may further comprise one or more storage devices, which may be embodied in a single memory device, a plurality of memory devices, and/or embedded circuitry of a device. Such data storage may comprise read-only memory, random access memory, volatile memory, non-volatile memory, static memory, dynamic memory, flash memory, cache memory, data storage registers, and/or any device that stores digital information. It should be noted that in embodiments in which control circuitry comprises a hardware and/or software state machine, analog circuitry, digital circuitry, and/or logic circuitry, data storage device(s)/register(s) storing any associated operational instructions may be embedded within, or external to, the circuitry comprising the state machine, analog circuitry, digital circuitry, and/or logic circuitry.

The control circuitry 211, 251 may comprise computer-readable media storing, and/or configured to store, hard-coded and/or operational instructions corresponding to at least some of the steps and/or functions illustrated in one or more of the present figures and/or described herein. Such computer-readable media can be included in an article of manufacture in some instances. The control circuitry 211/251 may be entirely locally maintained/disposed or may be remotely located at least in part (e.g., communicatively coupled indirectly via a local area network and/or a wide area network). Any of the control circuitry 211, 251 may be configured to perform any aspect(s) of the various processes disclosed herein.

With respect to the robotic system 10, at least a portion of the control circuitry 211 may be integrated with the base 25, column 14, and/or console 13 of the robotic system 10, and/or another system communicatively coupled to the robotic system 10. With respect to the control system 50, at least a portion of the control circuitry 251 may be integrated with the console base 51 and/or display unit 56 of the control system 50. It should be understood that any description herein of functional control circuitry or associated functionality may be understood to be embodied in the robotic system 10, the control system 50, or any combination thereof, and/or at least in part in one or more other local or remote systems/devices, such as control circuitry associated with a handle/base of a shaft-type instrument (e.g., endoscope) in accordance with any of the disclosed embodiments.

With further reference to FIG. 4-1, the control system 50 can include various I/O components 218 configured to assist the physician 5or others in performing a medical procedure. For example, the input/output (I/O) components 218 can be configured to allow for user input at input control(s) 255 to control/navigate the scope 40 and/or basketing system within the patient 7. In some embodiments, for example, the physician 5 can provide input to the control system 50 and/or robotic system 10, wherein in response to such input, control signals can be sent to the robotic system 10 to manipulate the scope 40 and/or catheter basketing system 30. The control system 50 can include one or more display devices 56 to provide various information regarding a procedure. For example, the display(s) 56 can provide information regarding the scope 40 and/or basketing system 30. For example, the control system 50 can receive real-time images that are captured by the scope 40 and display the real-time images via the display(s) 56. Additionally or alternatively, the control system 50 can receive signals (e.g., analog, digital, electrical, acoustic/sonic, pneumatic, tactile, hydraulic, etc.) from a medical monitor and/or a sensor associated with the patient 7, and the display(s) 56 can present information regarding the health or environment of the patient 7. Such information can include information that is displayed via a medical monitor including, for example, information relating to heart rate (e.g., ECG, HRV, etc.), blood pressure/rate, muscle bio-signals (e.g., EMG), body temperature, blood oxygen saturation (e.g., SpO₂), CO₂, brainwaves (e.g., EEG), environmental and/or local or core body temperature, and so on.

The various components of the system 100 can be communicatively coupled to each other over a network, which can include a wireless and/or wired network. Example networks include one or more personal area networks (PANs), local area networks (LANs), wide area networks (WANs), Internet area networks (IANs), cellular networks, the Internet, personal area networks (PANs), body area network (BANs), etc. For example, the various communication interfaces 254, 214 of the systems of FIG. 4-1 can be configured to communicate with one or more device/sensors/systems, such as over a wireless and/or wired network connection. In some embodiments, the various communication interfaces 254, 214 can implement a wireless technology such as Bluetooth, Wi-Fi, near-field communication (NFC), or the like. Furthermore, in some embodiments, the various components of the system 100 can be connected for data communication, fluid exchange, power exchange, and so on via one or more support cables, tubes, or the like.

The control system 50 and/or robotic system 10 can include certain user controls (e.g., controls 55), which may comprise any type of user input (and/or output) devices or device interfaces, such as one or more buttons, keys, joysticks, handheld controllers (e.g., video-game-type controllers), computer mice, trackpads, trackballs, control pads, and/or sensors (e.g., motion sensors or cameras) that capture hand gestures and finger gestures, touchscreens, and/or interfaces/connectors therefore. Such user controls are communicatively and/or physically coupled to respective control circuitry. In some embodiments, the user may engage the user controls 55 to command robotic shaft rotation/roll, as described herein.

The end effectors 22 may be configured to operate one or more adapters 8 (e.g., sterile adapters) which may be removably attached to the end effectors 22. While adapters 8 are shown as part of the robotic system 10, the adapters 8 may be separate devices which may be mated to one or more end effectors 22 to provide a sterile extension of the end effectors 22 for use in operating and/or driving one or more medical instruments. For example, a protective sheet 38 may be configured to separate at least a portion of the arms 12 and/or end effectors 22 from the adapters 8.

The adapters 8 may comprise one or more drive outputs 404 configured to translate force (e.g., torque) from the drive outputs 402 of the end effectors 22 to one or more medical instruments mated to the drive outputs 404 at the adapters 8. One or more adapters 8 may additionally or alternatively comprise one or more drive inputs configured to receive and/or otherwise mate with the drive outputs 402 of the end effectors 22.

FIG. 4-2 illustrates medical system components, including scope 519 and basketing 30 devices/assemblies 519 that may be implemented in any of the medical systems of FIGS. 1–3 in accordance with one or more embodiments. In some embodiments, the scope assembly 519 includes a handle or base 31 coupled to an endoscope 40. For example, the endoscope (i.e., “scope” or “shaft”) can include an elongate shaft including one or more lights 49 and one or more cameras or other imaging devices 48. The scope 40 can further include one or more working channels 44, which may run a length of the scope 40. In some embodiments, such channel(s) may be utilized to provide access for elongate basketing wires/tines through the scope 40.

The basketing assembly 30 can comprise a basket 35 formed of one or more wire tines 36. For example, the basketing assembly 30 may comprise four wire tines disposed within a basketing sheath 37 over a length thereof, wherein the tines project from a distal end of the sheath 37 to form the basket form 35. The tines 36 further extend from the proximal end of the sheath 37. The tines 36 may be configured to be slidable within the basketing sheath 37, subject to some amount of frictional resistance. The tines 36 and the sheath 37 can be coupled to respective actuators 195 of a basket cartridge component 32. The basket cartridge 32 may be physically and/or communicatively coupled to the handle portion/component 31 of the scope assembly 519. The handle component 31 can be configured to be used to assist in basketing and/or scope control either manually or through robotic control.

The basketing assembly 30 may comprise various interaction elements 411 which may allow the basketing assembly 30 to interact in various ways with the scope assembly 519 and/or components of the robotic system 10 (e.g., end effectors 22 and/or adapters 8). For example, the basketing assembly 30 may comprise one or more drive inputs 413 configured to receive and/or mate with corresponding drive outputs of one or more end effectors 22 and/or adapters 8. Example interactions elements 411 can further include alignment features 415 configured to facilitate alignment of the basketing assembly 30 with the scope assembly 519, an adapter 8, an end effector 22, and/or other device. For example, the basketing assembly 30 may comprise one or more magnetic elements configured to mate with corresponding magnetic elements at the scope assembly 519, adapter 8, and/or other device. The one or more alignment features 415 may be configured to mate with corresponding features only when the basketing assembly 30 is situated in a desired configuration with the scope assembly 519, adapter 8, and/or other device.

In some embodiments, the basketing assembly 30 may comprise one or more securing features 418 configured to secure the basketing assembly 30 to the scope assembly 519, adapter 8, and/or other device. For example, the basketing assembly 30 may comprise one or more hooks and/or similar attachments mechanisms configured to mate with and/or otherwise form an attachment with corresponding attachment mechanisms (e.g., latches) at an adapter 8. The basketing assembly 30 may further comprise a release mechanism to allow the basketing assembly 30 to be removed from the scope assembly 519, adapter 8, and/or other device.

The scope assembly 519 may comprise various interaction elements 401 which may allow the scope assembly 519 to interact in various ways with the basketing assembly 30 and/or components of the robotic system 10 (e.g., end effectors 22 and/or adapters 8). For example, the scope assembly 519 may comprise one or more drive inputs 403 configured to receive and/or mate with corresponding drive outputs of one or more end effectors 22 and/or adapters 8. Example interactions elements 401 can further include alignment features 405 configured to facilitate alignment of the scope assembly 519 with the basketing assembly 30, an adapter 8, an end effector 22, and/or other device. For example, the scope assembly 519 may comprise one or more magnetic elements and/or other adapter alignment features 406 configured to mate with corresponding magnetic elements and/or other features at an adapter 8. In another example, the scope assembly 519 may comprise one or more magnetic elements and/or other basket alignment features 407 configured to mate with corresponding magnetic elements and/or other features at the basketing assembly 30. The one or more alignment features 405 may be configured to mate with corresponding features only when the scope assembly 519 is situated in a desired configuration with the basketing assembly 30, adapter 8, and/or other device.

In some embodiments, the scope assembly 519 may comprise one or more securing features 418 configured to secure the scope assembly 519 to the basketing assembly 30, adapter 8, and/or other device. For example, the scope assembly 519 may comprise one or more indentations and/or similar attachments features configured to mate with and/or otherwise form an attachment with corresponding attachment mechanisms (e.g., latches) at an adapter 8. The scope assembly 519 may further comprise a release mechanism to allow the scope assembly 519 to be removed from the basketing assembly 30, adapter 8, and/or other device.

The scope assembly 519 can be powered through a power interface 45 and/or controlled through a control interface 78, each or both of which may interface with a robotic arm/component of the robotic system 10. The scope assembly 519 may further comprise one or more sensors 172, such as pressure and/or other force-reading sensors, which may be configured to generate signals indicating forces experienced at/by one or more of the actuators 195 and/or other couplings of the scope/basket system 519.

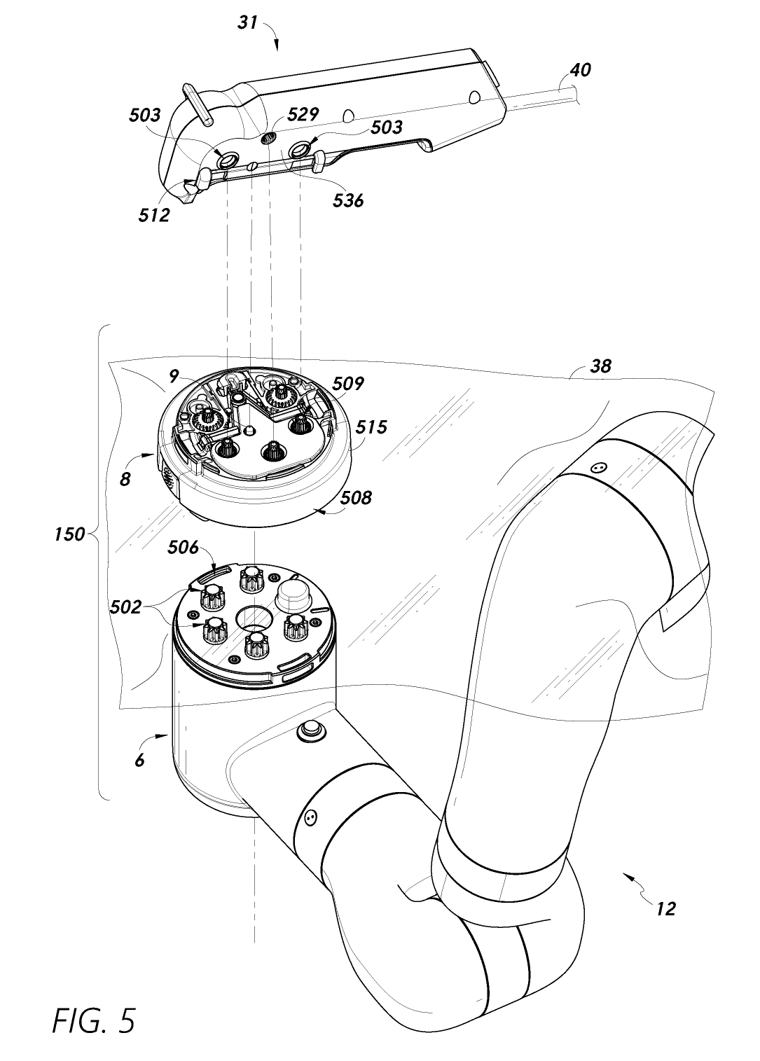

FIG. 5 shows an exploded view of an instrument device manipulator assembly 150 associated with a robotic arm 12 configured to drive at least a medical instrument 31 in accordance with one or more embodiments. The instrument device manipulator assembly 150 includes an end effector 6 associated with a distal end of the robotic arm 12. The instrument manipulator assembly 150 may be configured to manipulate drive inputs which may be associated with a handle portion of the medical instrument 31. Description herein of upward-facing and downward-facing surfaces, plates, faces, components, and/or other features or structures may be understood with reference to the particular orientation of the instrument device manipulator assembly 150 shown in FIG. 5, as assembled (rather than the tilted, exploded orientations shown). That is, although the end effector 6 may generally be configurable to face and/or be oriented in a range of directions and orientations, for convenience, description of such components (and components/devices attached/latched thereto directly or indirectly) herein may be in the context of the generally vertical facing orientation of the end effector 6 shown in FIG. 5.

In some embodiments, the instrument device manipulator assembly 150 further includes an adapter component 8 that is mountable to the end effector 6 and configured to provide a driver interface between the end effector 6 and the medical instrument 31. The adapter 8 and/or the medical instrument 31 may be removable or detachable from the robotic arm 12 and may be devoid of any electro-mechanical components, such as motors, in some embodiments. This dichotomy may be driven by the need to sterilize medical instruments used in medical procedures and the inability to adequately sterilize expensive capital equipment due to their intricate mechanical assemblies and sensitive electronics. Accordingly, the medical instrument 31 and/or adapter 8 may be designed to be detached, removed, and interchanged from the end effector 6 (and thus the system) for individual sterilization or disposal. In contrast, the end effector 6 need not be changed or sterilized in some cases and may be draped (e.g., using drape 38) for protection.

In some embodiments, the adapter 8 can include connectors to transfer pneumatic pressure, electrical power, electrical signals, and/or optical signals from the robotic arm 12 and/or end effector 6 to the medical instrument 31 and/or to additional instruments. The robotic arm 12 can advance/insert or retract the coupled medical instrument 31 into or out of the treatment site. In some embodiments, the medical instrument 31 can be removed and replaced with a different type of instrument and/or can be supplemented with additional instruments. The end effector 6 of the robotic arm 12 can include various components/elements configured to connect to and/or align with components of the adapter 8, instrument handle, and/or shaft 40. For example, the end effector 6 can include drive outputs 502 (e.g., drive splines, gears, or rotatable disks with engagement features) to control/articulate a medical instrument and/or one or more fasteners 506 to attach the medical instrument 31 and/or adapter 8 to the end effector 6. In some embodiments, a portion (e.g., plate) 515 of the adapter 8 can be configured to rotate/spin independently of one or more other components of the adapter 8 and/or end effector 6 when coupled to the end effector 6.

In some configurations, a sterile drape 38, such as a plastic sheet or the like, may be disposed between the end effector 6 and the adapter 8 to provide a sterile barrier between the robot arm 12 and the instrument handle 31. For example, the drape 38 may be coupled to the adapter 8 in such a way as to allow for translation of mechanical torque from the end effector 6 to the adapter 8. The adapter 8 may generally be configured to maintain a seal around the actuating components thereof, such that the adapter 8 provides a sterile barrier itself. The use of a drape 38 coupled to the adapter 8 and/or more other component(s) of the device manipulator assembly 150 may provide a sterile barrier between the robotic arm 12 and the surgical field, thereby allowing for the use of the robotic cart associated with the arm 12 in the sterile surgical field. The end effector 6 may be configured to be coupled to various types of sterile adapters that may be loaded onto and/or removed from the end effector 6 of the robotic arm 12. With the arm 12 draped in plastic, the physician and/or other technician(s) may interact with the arm 12 and/or other components of the robotic cart (e.g., screen) during a procedure. Draping may further protect against equipment biohazard contamination and/or minimize clean-up after procedure. The adapter 8 may comprise a base portion 508 configured to provide a foundation for various components of the adapter 8.

The medical instrument 31 can include a plurality of drive inputs 503, 529 on a lower surface 536 of the housing of the instrument handle 31. In the illustrated embodiment, the medical instrument 31 includes three drive inputs 503, 529, although other numbers of drive inputs can be included in other embodiments. The drive inputs can be in fixed positions spaced apart along the lower mating surface 536 of the medical instrument 31, which facilitates coupling the drive inputs 503, 529 to the corresponding drive outputs 502 of the end effector 6 and/or drive outputs of the adapter 8, which may be in fixed positions spaced apart along a corresponding mating surface designed for modular use and attachment to a variety of other instruments. The instrument 31 can include latching clips 512 or other latching features/means for physically coupling to corresponding structure of the adapter 8 and/or end effector 6.

Other example instruments that can be manipulated via the device manipulator assembly can include robotically controlled catheters, EM field generators, retrieval basket tools, laser fiber drivers, and/or distal drive devices, among others.

References herein to an “instrument device manipulator assembly,” “instrument manipulator assembly,” “manipulator,” “manipulator assembly,” as well as other variations thereof, can refer to any subset of the components of the assembly 150 shown in FIG. 5, including a robot arm, an end effector of a robot arm, an adapter configured to be coupled to a robotic end effector, an instrument base/handle configured to be coupled to an end effector and/or adapter, and/or other actuator component(s), means, and/or mechanism associated with an instrument base/handle. Furthermore, it should be understood that references herein to an “actuator” can refer to any component of the assembly 150 shown in FIG. 5 that affects or causes, either directly or indirectly, movement of an instrument/component engaged with, coupled to, or otherwise actuatable by, a component of the assembly 150. For example, in accordance with embodiments disclosed here, an “actuator” may comprise any set or subset of the following devices or components: instrument feeder drive input(s), adapter drive output(s), adapter drive input(s), pulleys, belts, gears, pegs, pins, end effector drive output(s), and/or structures and/or control circuitry configured to cause actuation of the same. For example, an actuator may be any component, device, or structure configured such that movement thereof causes corresponding movement in another component, device, or structure, whether integrated with or separate from the actuator.

The adapter 8 may comprise one or more drive outputs 509 configured to translate torque from one or more drive outputs 502 of, for example, an end effector of a robotic system, to corresponding drive inputs 503 of one or more medical instruments. The end effector 6 and/or robotic system may comprise one or more drive outputs 502 configured to mate with corresponding drive inputs of the adapter 8. For example, each drive output 509 at the adapter 8 may be associated with a corresponding drive input (e.g., at an underside of the drive output 502) and/or may be configured to translate force (e.g., torque) from the robotic system. The adapter 8 may be configured to be removably attached to the robotic system. In some cases, a drape 38 may be configured to be situated at least partially between at least a portion of the robotic system and the adapter 8.

The one or more drive outputs 509 of the adapter 8 may be configured to translate movement and/or force of one or more drive outputs 502 of a robotic system to one or more medical instruments 31 which may be removably attached to the drive outputs 509 of the adapter 8. The one or more medical instruments 31 may be configured to mate with and/or attach to one or more drive outputs 509 of the adapter 8. For example, the medical instrument 31 (e.g., a scope device) illustrated in FIG. 5 may be configured to mate with one or more drive outputs 509 of the adapter 8. However, various types of medical instruments may be configured to be driven by the adapter 8 and/or an associated robotic system. Moreover, while only a scope-type medical instrument 31 is shown in FIG. 5, the adapter 8 may be configured for driving multiple medical instruments 31 and/or various types of medical instruments 31.

While only a single adapter 8 is shown in FIG. 5, multiple adapters 8 may be utilized simultaneously and/or may be configured for use with multiple drivers (e.g., multiple robotic arms 12) of a drive system. For example, as shown in FIG. 4-1 a robotic system may comprise three robotic arms 12. In such cases, one, two, or three adapters 8 may be used to adapt to the three robotic arms 12.

The adapter 8 may be configured for facilitating attachment of one or more medical instruments 31 with the adapter 8 before, during, and/or following a medical procedure. For example, the adapter 8 may be configured to facilitate docking of one or more medical instruments 31 while the one or more medical instruments is in use and/or after the medical instrument 31 has been delivered into a patient’s body. To minimize the risk of harm to the patient, the adapter 8 and/or medical instrument 31 may be configured to simplify and/or facilitate docking of the medical instrument 31 at the adapter 8 using various means which will be described herein. In this way, docking of the medical instrument 31 at the adapter 8 may require minimal attention from a physician guiding the medical instrument at the patient’s body.

In some embodiments, transfer of force from the end effector 6 to the adapter 8 and/or from the adapter 8 to the medical instrument 31 may involve ultra-low friction input transfer. Translation of force may involve mesh engagement between one or more drive outputs and/or one or more drive inputs. For example, teeth of a gear at a drive output may be configured to mesh with and/or fit into corresponding receptors at a drive input.

### Instrument Docking

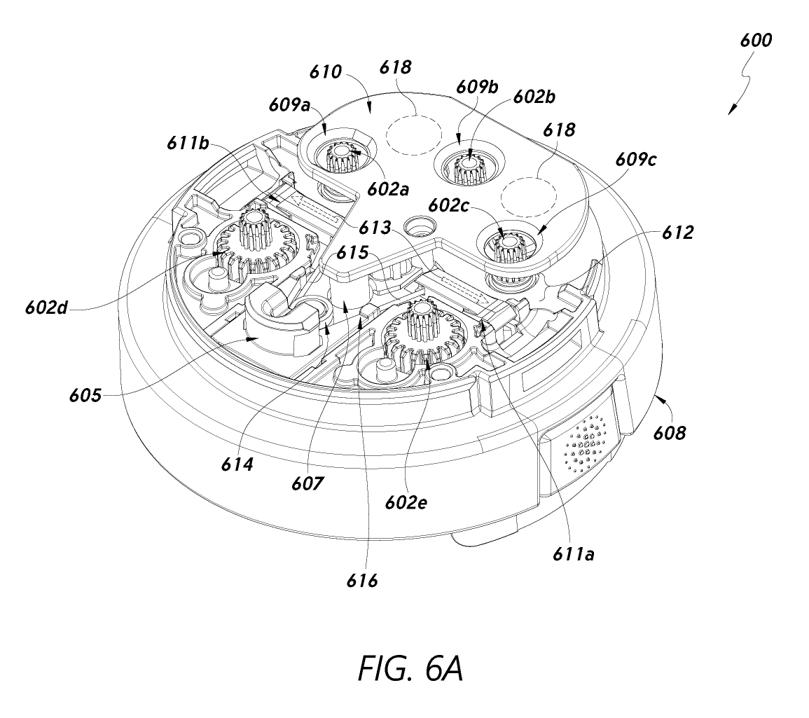

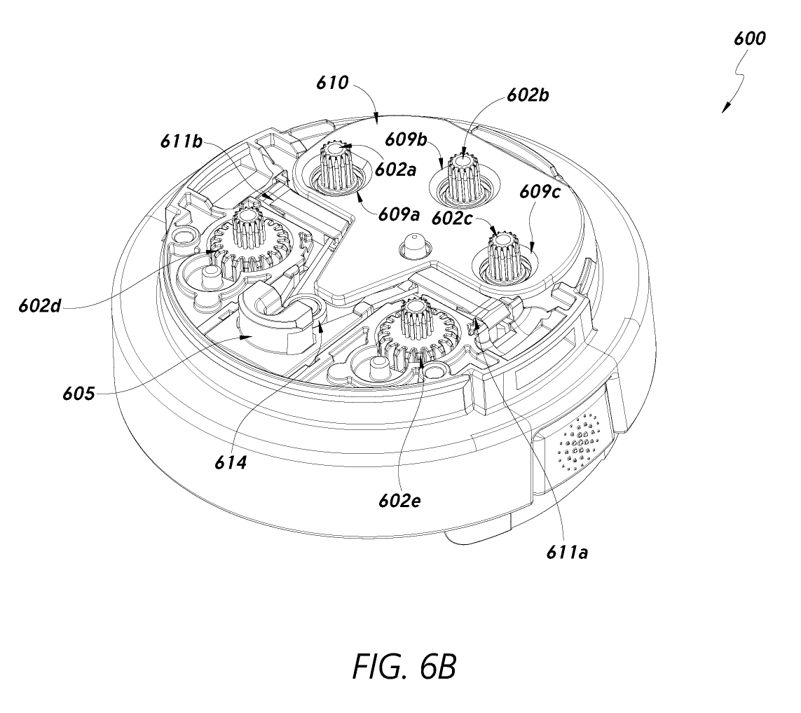

FIGS. 6A and 6B illustrate at least a portion of a drive assembly 600, which may include one or more adapters, having features configured to facilitate docking of one or more medical instruments. In some embodiments, the drive assembly 600 may comprise a platform 610 extending from, attached to, and/or secured to a base portion 608 of the drive assembly 600. In some embodiments, the platform 610 may be configured to move between multiple configurations without becoming detached and/or disconnected from the base portion 608. The base portion 608 may provide a foundation and/or base for various components of the drive assembly 600. For example, the platform 610, one or more drive outputs 602, release mechanism 605, torsion spring 614, and/or latching mechanisms may be configured to extend from the base portion 608. However, such components and/or other components of the drive assembly 600 may be configured to extend from other components and/or may not be directly coupled to the base portion 608. In some embodiments, various components of the drive assembly 600 (e.g., the platform 610) may comprise extensions of the base portion 608.

The platform 610 may be configured to be movable between an elevated (e.g., raised) configuration (shown in FIG. 6A) and a depressed (e.g., lowered) configuration (shown in FIG. 6B). In the elevated configuration, the platform 610 may be configured to receive and/or guide one or more medical instruments. The platform 610 and/or base portion 608 may have any of a variety of features configured to facilitate attachment and/or docking of one or more medical instruments at the platform 610. For example, the platform 610 may comprise one or more magnetic elements configured to mate with corresponding magnetic elements of one or more medical instruments.

The drive assembly may comprise one or more docking portions configured to receive one or more medical instruments. As used herein, the term “docking portion” is used in accordance with its plain and ordinary meaning and may refer to any portion of a drive assembly 600 including any number of components of the drive assembly 600 that may be configured to receive one or more medical instruments by, for example, forming a removable and/or secure attachment to the one or more medical instruments and/or accommodating placement and/or attachment of the one or more medical instruments at and/or to the drive assembly 600. In some embodiments, the platform 610 and/or a first set of drive outputs may be associated with and/or comprise at least a portion of a first docking portion and/or may be configured to receive one or more medical instruments by allowing placement of one or more medical instruments at the platform 610, attaching to one or more medical instruments using magnetic elements and/or other features, and/or forming a secure attachment to one or more medical instruments attached via latching mechanisms 611 of the drive assembly 600. A second set of drive outputs (e.g., a fourth drive output 602d and/or a fifth drive output 602e) and/or a release mechanisms 605 may be associated with and/or comprise at least a portion of a second docking portion and/or may be configured to receive one or more medical instruments by mating the second set of drive outputs with the one or more medical instruments and/or allowing the one or more medical instruments to attach and/or latch to the release mechanism 605.

In some embodiments, the platform 610 may be biased in the elevated configuration shown in FIG. 6A. Thus, the platform 610 may be configured to naturally extend at least a first distance 612 from the base portion 608. For example, one or more springs (not shown in FIGS. 6A and 6B) may extend from the base portion 608 and/or platform 610 and/or may otherwise be situated at least partially between the platform 610 and the base portion 608 (e.g., extending at least partially into openings of the base portion) to press the platform 610 to the elevated configuration. In the elevated configuration, a gap 612 between the platform 610 and the base portion 608 may be greater than the gap between the platform 610 and the base portion 608 in the in the depressed configuration.

The platform 610 may be associated with and/or situated adjacent to one or more drive outputs 602 coupled to and/or extending from the base portion 608 of the drive assembly 600. The one or more drive outputs 602 may be configured to drive one or more medical instruments. One or more of the one or more drive outputs 602 may be configured to drive a single type of medical instrument and/or may be configured to drive multiple types of medical instruments. For example, a drive output 602 may be configured to mate with a first type of drive input associated with a first type of medical instrument and/or with a second type of drive input associated with the first type of medical instrument and/or a second type of medical instrument. In some embodiments, the drive assembly 600 may comprise a first set of one or more drive outputs 602 (e.g., including a first drive output 602a, a second drive output 602b, and a third drive output 602c) configured to drive a first medical instrument and/or a first type of medical instrument and/or the drive assembly 600 may comprise a second set of one or more drive outputs 602 (e.g., including a fourth drive output 602d and a fifth drive output 602e) configured to drive a second medical instrument and/or a second type of medical instrument.

In some embodiments, the platform 610 may comprise one or more apertures 609, which may include openings, cavities, gaps, grooves, indentations, and/or similar features, configured to accommodate at least a first set of the one or more drive outputs 602. As shown in the example shown in FIGS. 6A and 6B, the platform 610 may comprise three apertures 609 (i.e., a first aperture 609a, a second aperture 609b, and a third aperture 609c) with each aperture 609 configured to receive a different drive output 602. However, the platform 610 may comprise any number of apertures 609 and/or may be configured to accommodate any number of drive outputs 602. Moreover, while the apertures 609 and drive outputs 602 are shown having a circular form, the apertures 609, drive outputs 602, and/or other features of the drive assembly 600 may have different shapes and/or sizes. For example, the platform 610 may comprise a single aperture 609 configured to receive multiple drive outputs 602. Such an aperture 609 may have a generally elongated form to extend between drive outputs 602.

In the depressed configuration shown in FIG. 6B, one or more drive outputs 602 may extend through and/or beyond the platform 610. When the platform 610 extends to the elevated configuration shown in FIG. 6A, the platform 610 may extend beyond at least a portion of the drive outputs 602. For example, the platform 610 and/or apertures 609 may be suspended above at least a portion of one or more drive outputs 602 and/or may be situated generally in-line with distal tip portions of the drive outputs while in the elevated configuration. However, in some embodiments, distal tip portions of the drive outputs 602 may extend at least partially beyond the platform 610 in the elevated configuration.

In some embodiments, the platform 610 and/or base portion 608 may comprise various features configured to guide (e.g., funnel, direct, move) one or more medical instruments to the platform 610. For example, one or more magnetic elements may be situated at a surface of the platform 610, within the platform 610, and and/or beneath the platform 610 (e.g. between the platform 610 and the base portion 608). In some embodiments, the platform 610 and or base portion 608 may comprise a first magnetic element having a first polarity and/or a second magnetic element having a second polarity such that the first and second magnetic elements may be configured to align the one or more medical instruments with the platform 610. For example, the magnetic elements may have suitable polarities and/or may be situated such that one or more medical instruments may be configured to mate with the magnetic elements in only a single configuration. FIGS. 6A and 6B include dotted circles 618 indicating example positions of one or more at magnetic elements at or below the platform. However, the magnetic elements may have any shape and/or size. Moreover, while three magnetic elements are indicated in FIGS. 6A and 6B, any number of magnetic elements may be used.

When the platform 610 is in the elevated configuration shown in FIG. 6A, the platform 610 may be configured to be pressed down toward the base portion 608 to move the platform 610 to the depressed configuration shown in FIG. 6B. Relatively little force may be required to press the platform into the depressed configuration (in other words, to overcome bias from one or more biasing elements, such as springs). For example, a physician may situate a first medical instrument above the platform 610. One or more guiding features at the platform 610 and/or base portion 608 may be configured to guide the first medical instrument into a proper configuration and/or attachment at the platform 610. With the first medical instrument in place with respect to the platform 610, the physician may press the first medical instrument and/or platform 610 toward the base portion 608. The platform 610 may be configured to attach to one or more medical instruments. In some embodiments, an attachment between the platform 610 and one or more medical instruments may be a breakable attachment configured to be overcome using a relatively small amount of force (e.g., two Newtons).

The drive assembly 600 may comprise any of a variety of suitable features for holding the platform 610 in the depressed configuration and/or for maintaining an attachment between one or more medical instruments and the platform 610 while the platform is in the depressed configuration. As shown in FIG. 6B, the drive assembly 600 may comprise one or more latching mechanisms 611 (e.g., a first latching mechanism 611a and/or a second latching mechanism 611b) configured to hold one or more medical instruments against the platform 610 while the platform 610 is in the depressed configuration. The term “latching mechanism” is used herein in accordance with its plain and ordinary meaning and may include any device, element, feature, and/or component configured to latch and/or lock an object in place and/or to hold multiple objects together. In some embodiments, the one or more latching mechanisms 611 may be configured to cause the platform 610 to be held in the depressed configuration. The latching mechanisms 611 may be configured to directly hold the platform 610 in the depressed configuration and/or to indirectly hold the platform 610 and/or to cause the platform to be held in the depressed configuration by holding a medical instrument docked at the platform 610 in place. The drive assembly 600 may further comprise a release mechanism 605 configured to release the latching mechanisms 611 and/or to otherwise allow the platform 610 to return to the elevated configuration and/or to allow the one or more medical instruments to separate from the platform 610. The platform 610 may comprise one or more engagement features 607 (e.g., having a form of a hollow cylindrical column) configured to extend along corresponding lumens of the base portion 608.

In some embodiments, the one or more latching mechanisms 611 may be biased in outward 613 direction at least in part by a torsion spring and/or similar device. The torsion spring may comprise a coiled portion 614 and/or one or more elongate ends 615 which may be configured to press the one or more latching mechanisms 611 in the outward 613 direction. The release mechanism 605 may comprise one or more arm portions 616 which may press the elongate ends 615 of the torsion spring inwards (e.g., opposite the biased direction) when the release mechanism 605 is pressed against the torsion spring and/or moved towards the platform 610. Moreover, a medical instrument may be configured to press the one or more latching mechanisms inward during a docking process of the medical instrument.

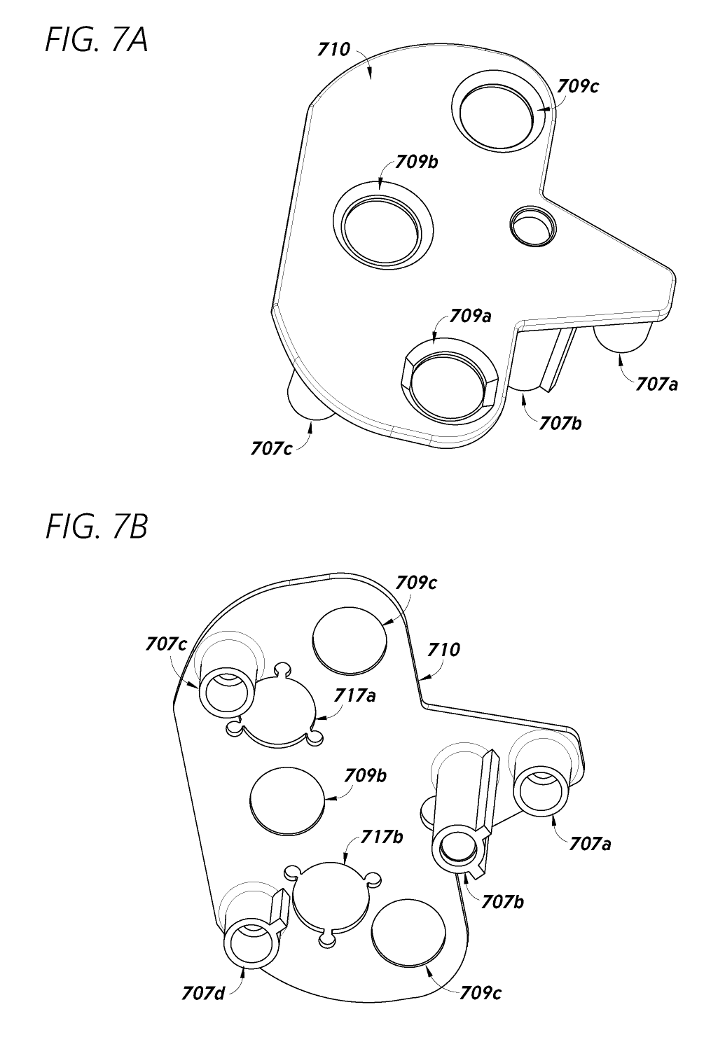

FIGS. 7A and 7B provide illustrations of example platforms 710 for use with one or more drive assemblies. A platform 710 may comprise various drive output receiving features, which may include apertures 709. As shown in FIG. 7B, a platform 710 may comprise receptors 717 for medical instrument guiding features (e.g., magnetic elements). While FIG. 7B shows receptors 717 having a generally circular form, suitable receptors 717 may have any shape and/or size.

The platform may further comprise various engagement features 707 (e.g., a first engagement feature 707a, a second engagement feature 707b, and/or a third engagement feature 707c) for engaging with the base portion and/or other elements of the drive assembly. For example, the one or more engagement features 707 may have generally cylindrical shapes and/or may be configured to extend into corresponding openings (e.g., lumens) of a base portion to allow the platform 710 to extend along the openings of the base portion. Moreover, the engagement features 707 may be configured to maintain a connection between the platform 710 and a base portion while the platform 710 moves between the elevated configuration and the depressed configuration.

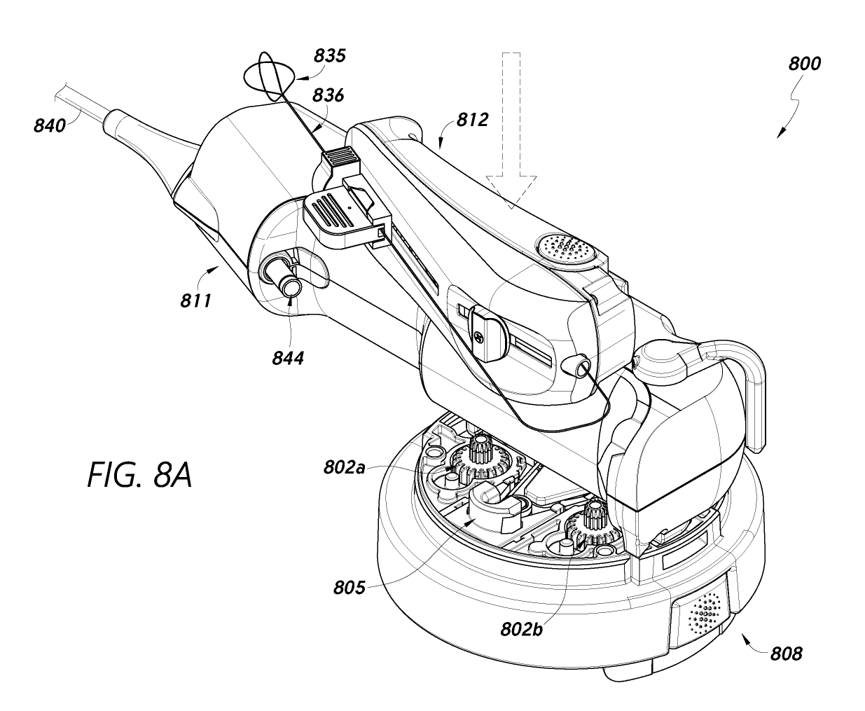

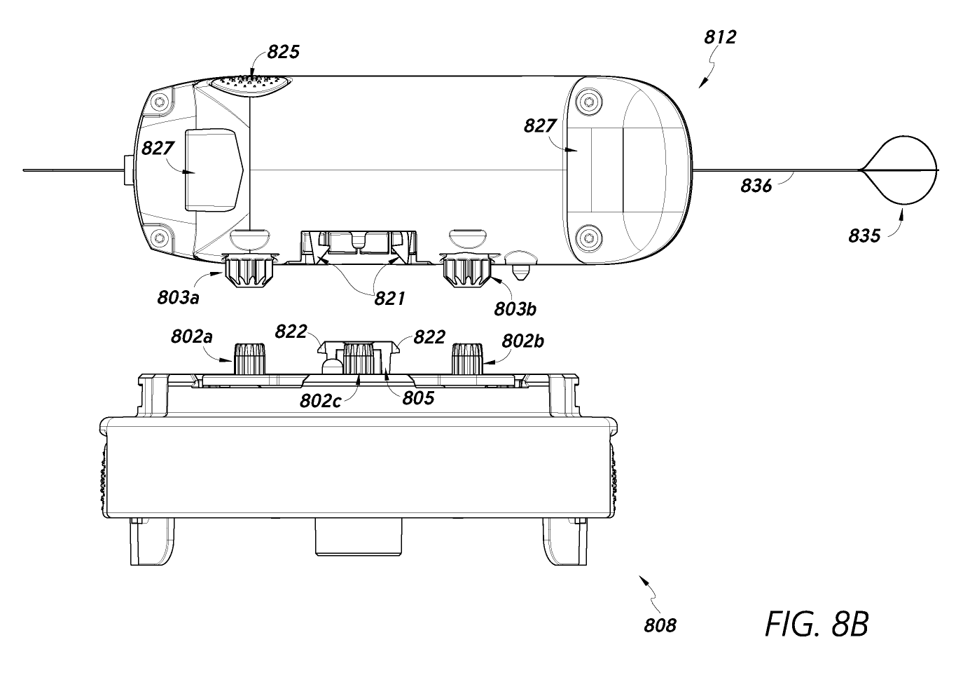

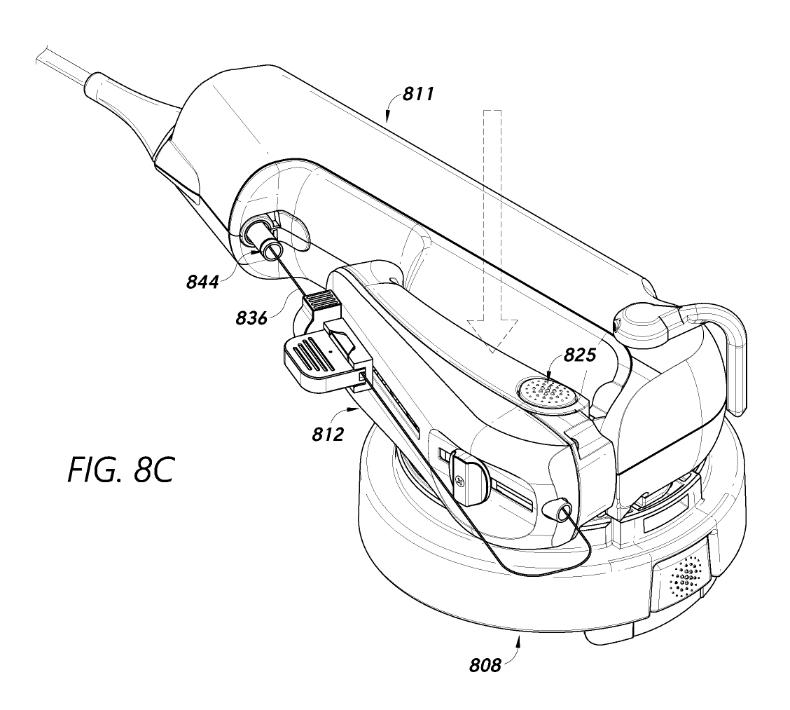

FIGS. 8A-8C illustrate a drive assembly 800 including an adapter 808 configured to drive one or more medical instruments, which can include a first medical instrument 811 and/or a second medical instrument 812. The first medical instrument 811 and the second medical instrument 812 may be configured to operate independently and/or in combination. For example, the first medical instrument 811 may be a scoping device (e.g. a ureteroscope) and/or the second medical instrument 812 may be a working channel tool (e.g. a basketing tool) configured to be operated in combination with the scoping device. While the first medical instrument 811 is illustrated as a scope device and the second medical instrument 812 is illustrated as a working channel tool in FIGS. 8A-8C, different types of medical instruments may be used and/or may be configured to be driven by the adapter 808.

In some embodiments, the first medical instrument 811 may be configured to operatively receive at least a portion of the second medical instrument 812. For example, the first medical instrument 811 may comprise a receptor 844, which can include a lumen, chamber, and/or other feature, configured to receive at least a portion of the second medical instrument 812. The second medical instrument 812 may comprise one or more tines 835 and/or a sheath 836 extending from the second medical instrument 812. The one or more tines 835 and/or sheath 836 may be configured to be operated via drive inputs at the second medical instrument 812. The tines 835 may a medical tool configured to extend out of a distal end portion of the sheath 836. As shown in FIGS. 8A-8C, the medical tool may include a basket tool. The one or more tines 835 and/or sheath 836 may be configured to fit at least partially into the receptor 844 of the first medical instrument 811 when the first medical instrument 811 and/or the second medical instrument 812 is/are docked at the drive assembly adapter 808.

The first medical instrument 811 and/or the second medical instrument 812 may comprise one or more features for facilitating an attachment between the first medical instrument 811 and the second medical instrument 812. For example, the second medical instrument 812 may comprise one or more engagement features, such as magnetic elements, which may be configured to fit into slots 827 of the second medical instrument 812. The one or more engagement features at the second medical instrument 812 may be configured to engage with corresponding features at the first medical instrument 811. For example, the second medical instrument 812 and/or first medical instrument 811 may comprise one or more magnetic elements configured to mate with each other when the first medical instrument 811 and/or the second medical instrument 812 is/are docked at the adapter 808. In this way, the first medical instrument 811 and/or second medical instrument 812 may be prevented from rolling and/or otherwise moving out of a desired position/orientation.

The first medical instrument 811 may be configured to dock at and/or attach to a first docking portion of the adapter 808. For example, the first medical instrument may be configured to attach to a movable platform at the first docking portion of the adapter 808. The first docking portion may comprise one or more drive outputs 802 configured to receive and/or drive one or more drive inputs of the first medical instrument (see, e.g. FIG. 5). The adapter 808 may additionally or alternatively comprise a second docking portion configured to receive the second medical instrument 812. The second docking portion may comprise one or more drive outputs 802 (e.g., a first drive output 802a and/or a second drive output 802b) configured to drive one or more drive inputs at the second medical instrument 812. The first medical instrument 840 may comprise a shaft 840 configured to provide access to a patient’s body.

The adapter 808 may comprise one or more latches (see, e.g., the latching mechanisms 611 of FIGS. 6A and 6B) configured to secure the first medical instrument at the drive assembly. For example, the one or more latches may be configured to fit into corresponding receptors of the first medical instrument 811 to prevent the first medical instrument 811 from becoming displaced from the adapter 808. The adapter 808 may additionally or alternatively comprise a release device 805 (e.g., a release mechanism and/or release) configured to release the one or more latches to allow the first medical instrument 811 to be removed from the adapter 808. In some embodiments, the release device 805 may be situated at least partially at or near the second docking portion of the drive assembly. For example, the release device 805 may be situated such that the second medical instrument 812, when docked at the second docking portion of the adapter 808, at least partially covers and/or prevents activation of the release device 805. In some embodiments, attaching the first medical instrument 811 at the first docking portion may involve pressing the first medical instrument 811 against the latches such that the latches are pressed inwards (e.g., away from an outer portion of the adapter 808 and/or against a bias of the latches by one or more torsion springs and/or similar mechanisms). The first medical instrument 811 may continue to press the first medical instrument 811 downwards until the latches enter one or more receptors, which can include grooves, indentations, cavities, etc., and/or the latches re-assume a biased outwards position to lock the first medical instrument 811 in place.

In some embodiments, the one or more latches may be biased outwardly toward an outer ring of the adapter 808 by one or more biasing mechanisms which can include torsion spring devices (see, e.g., the torsion spring devices 614, 615 of FIGS. 6A and 6B). For example, a torsion spring may be configured to press a first latch at a first side of the release device 805 and/or a second latch at a second side of the release device 805 outward toward the outer ring of the adapter 808 to engage the first latch and/or second latch. The release device 805 may be configured to be movable along the adapter 808. In some embodiments, the release device 805 may be situated such that moving the release device 805 toward the first docking portion (e.g., towards a platform of the adapter 808) causes the release device 805 to press against at least a portion of the torsion spring (e.g., one or more arms of the torsion spring) to cause one or more arms of the torsion spring to move inward, thus causing the one or more latches to move inward and/or away from the outer ring of the assembly 800 and/or to disengage to the one or more latches. For example, moving the release device 805 towards the first docking portion may cause compression at the torsion spring to remove an outward bias of the torsion spring.

The second medical instrument 812 may comprise one or more attachment devices 821 configured to attach to the release device 805 and/or one or more areas of the first docking portion. For example, as shown in FIG. 8B, a second medical instrument 812 may comprise one or more hooks 821 configured to grasp one or more latches 822 and/or protrusions at the release device 805 and/or other areas of the first docking portion. The second medical instrument 812 may further comprise a release button 825 configured to disengage the one or more attachment devices 821 to allow the second medical instrument 812 to disengage from the release device 805 and/or the second docking portion. Following removal of the second medical instrument 812 from the second docking portion, the release device 805 may be activated to allow removal of the first medical instrument 812 from the first docking portion. In this way, the release device 805 and/or the second medical instrument 812 may advantageously prevent the first medical instrument 811 from being removed from the adapter 808 until after the second medical instrument 812 is removed from the adapter 808. The second medical instrument 812 may comprise one or more drive inputs 803 configured to mate with corresponding drive outputs 802 of the adapter 808.

As shown in FIG. 8C, the first medical instrument 811 and second medical instrument 812 may be configured to be situated generally side-by-side while docked to the adapter 808. The first medical instrument 811 and the second medical instrument 812 may be configured to be docked at the adapter 808 simultaneously. Moreover, one or more drive outputs 802 of the adapter 808 may be configured to simultaneously operate the first medical instrument 811 and the second medical instrument 812. The sheath 836 extending from the second medical instrument 812 may be configured to be situated at least partially within the receptor 844 of the first medical instrument 811 while the second medical instrument 812 is docked at the adapter 808.

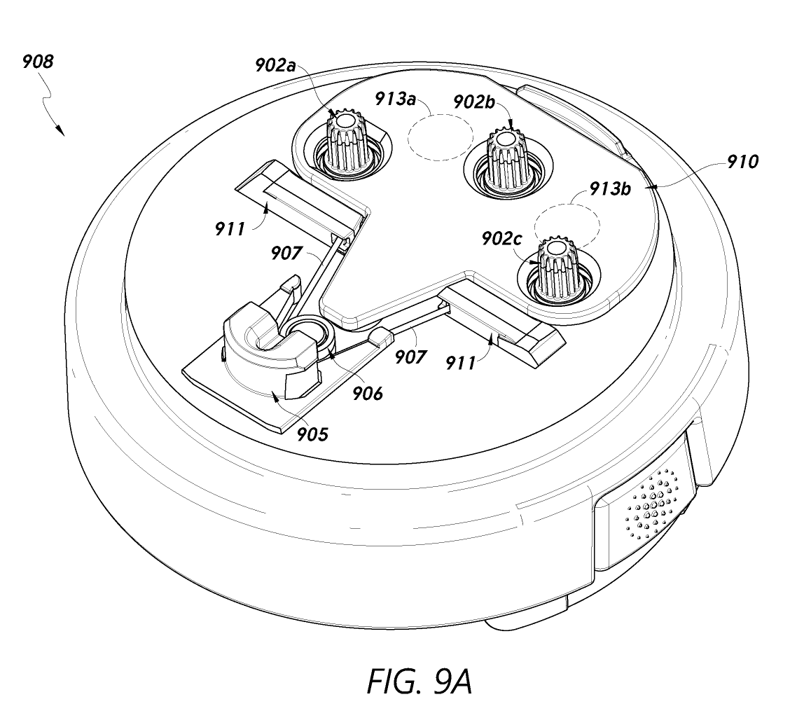

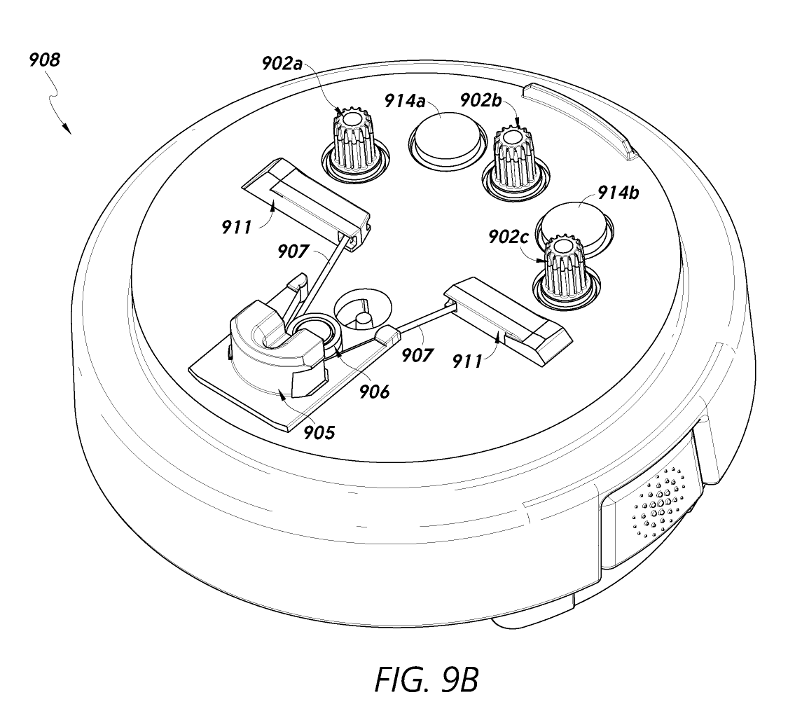

FIGS. 9A and 9B illustrate at least a portion of a drive assembly including an adapter 908 comprising one or more magnetic elements 914 and/or other alignment features configured to guide and/or attached to one or more medical instruments. The one or more magnetic elements 914 may be attached (e.g., glued) to and/or otherwise associated with a platform 910 and/or a first docking portion comprising and/or associated with the platform 910. In some embodiments, the platform 910 may extend from the adapter and/or the first docking portion of the adapter 908. The adapter 908 may comprise a first docking portion, which may include a first drive output 902a, a second drive output 902b, a third output 902c, and/or at least a portion of the platform 910, configured to receive a first medical instrument. The first medical instrument may be any suitable medical instrument, which can include scope devices as discussed herein. A first set of one or more drive outputs 902 (e.g., the first drive output 902a, the second drive output 902b, and/or the third output 902c) may extend from and/or at the first docking portion and/or may be configured to drive the first medical instrument when docked at the first docking portion. The adapter 908 may comprise a release mechanism 905 configured to interact with a torsion spring (e.g., comprising a coil portion 906 and/or one or more elongate ends 907) to control one or more latching mechanisms 911.

While magnetic elements 914 are described herein for illustrative purposes, other alignment features may be substituted for magnetic elements 914. In some embodiments, one or more magnetic elements 914 situated at and/or otherwise associated with the first docking portion may be configured to guide and/or establish alignment between a first medical instrument and the first docking portion and/or one or more drive outputs 902 associated with the first docking portion. For example, the one or more magnetic elements 914 may be configured to attract corresponding magnetic elements at the first medical instrument when the first medical instrument is placed within a magnetic field of the one or more magnetic elements 914. Accordingly, a physician may advantageously be enabled to dock the first medical instrument at the first docking portion by simply situating the first medical instrument within a general area of the first docking portion and/or may not be required to physically align the first medical instrument with the first set of drive outputs 902.

Two dashed circles 913 illustrated in FIG. 9B illustrate example positions of two magnetic elements 914 at or below the platform 910 and/or other portion of the first docking portion. However, a single magnetic element 914 and/or more than two magnetic elements 914 may be used. Moreover, the one or more magnetic elements 914 may have any suitable size and/or shape and may not necessarily have the circular shape shown in FIGS. 9A and 9B. FIG. 9B provides a cutout view of the drive assembly to illustrate the magnetic elements 914 below a surface of the platform 910 and/or other portions of the first docking portion. A first magnetic element 914a may have a first polarity and a second magnetic element 914b may have a second polarity that is different from the first polarity. Accordingly, the first and second magnetic elements 914 may be configured to cause a first medical instrument to dock at the first docking portion in only a single configuration, in which the magnetic elements 914 are aligned with corresponding magnetic elements at the first medical instrument. For example, a first magnetic element 914a may be configured to mate with a first magnet at a first medical instrument and/or a second magnetic element 914b may be configured to mate with a second magnet at the first medical instrument. The magnetic elements 914 may be configured to generate a magnetic field sufficient to attract one or more medical instruments when the medical instruments are not in contact with the platform 910. The magnetic elements 914 and/or platform 910 may be situated and/or configured such that lowering the platform 910 to the depressed configuration with the first medical instrument attached may cause one or more drive outputs of the adapter to extend into and/or otherwise engage with corresponding drive inputs of the first medical instrument.

The adapter 908 may be configured to receive additional medical instruments. For example, while the adapter 908 illustrated in FIGS. 9A and 9B comprises three drive outputs 902, the adapter 908 may comprise additional drive outputs 902 configured to receive a second medical instrument. Moreover, while magnetic elements 914 are shown at the first docking portion, the adapter 908 may comprise additional magnetic elements 914 and/or other guiding elements configured to guide docking of the second medical instrument.

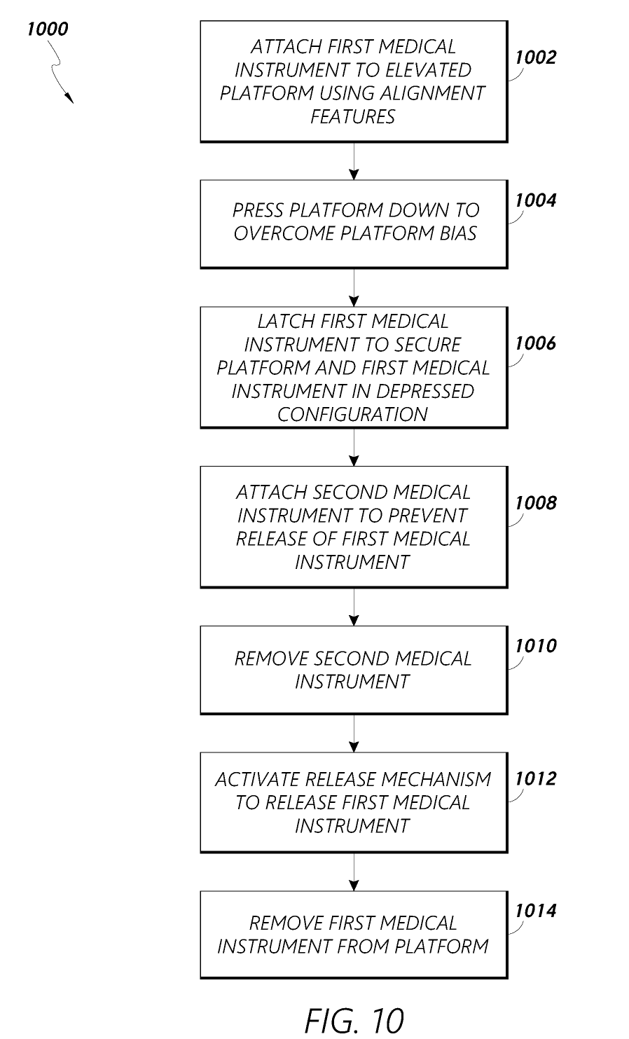

FIG. 10 provides a flow diagram for a process 1000 for driving one or more medical instruments at a drive assembly which may comprise one or more adapters (e.g., sterile adapters) for transferring force from a robotic system in accordance with one or more embodiments.

The process 1000 may be implemented in connection with a medical procedure, such as a kidney stone removal procedure, or other procedure that may be implemented using one or more medical instruments, such as endoscopes, ureteroscopes, EM field generators, basketing tools, laser fiber drivers, or the like. In some embodiments, the process 1000 may be implemented at least in part following placement of a distal end of a shaft of a first medical instrument (e.g., endoscope) within certain target anatomy of a patient, such as a calyx network of a kidney of the patient. One or more operations of the process 1000 may be implemented prior to access of the target anatomy by the distal end of the shaft of the first medical instrument.

At block 1002, the process 1000 involves attaching and/or docking a first medical instrument at an adapter of a drive assembly. In some embodiments, attaching, and/or docking the first medical instrument at the adapter may be facilitated at least in part via one or more alignment features (e.g., magnetic elements) at the adapter, as described herein. The one or more alignment features may be configured to form a breakable attachment between the first medical instrument and the adapter. For example, application of approximately 2 Newtons of pulling force may be sufficient to detach the first medical instrument from the platform. The one or more alignment features may advantageously allow physicians to focus on navigating the first medical instrument through a patient’s body to prevent and/or minimize damage to the kidneys and/or other tissue which may be caused imprecise navigation of the first medical instrument.

The first medical instrument may be configured to be docked and/or attached at least partially at a platform of the adapter. In some embodiments, the platform may extend from a base portion of the adapter. The first medical instrument may extend beyond the surface of the platform in at least some places.

The platform may be movable between two or more positions with respect to the base portion and/or other portions of the adapter. For example, the platform may be biased a first distance apart from the base portion such that the platform may be situated in a relatively elevated position when not acted upon by outside forces. The platform may be biased in the elevated position at least in part by one or more springs configured to press against a lower surface of the platform (e.g., between the platform and the base portion).

The adapter may comprise one or more drive outputs configured to drive corresponding drive inputs of the first medical instrument. The first medical instrument may be configured to attach and/or dock to the platform in a configuration in which at least some of the drive inputs of the first medical instrument are generally aligned with at least some of the drive outputs of the adapter. In some embodiments, the drive inputs of the first medical instrument may be suspended above the drive outputs when the first medical instrument is attached to the platform in the elevated position.

At step 1004, the process 1000 involves pressing the platform down overcome a bias of the platform. In some cases, the platform may be pressed down by a physician. The platform may be pressed down by pressing down on the first medical instrument while the first medical instrument is in contact with the platform. Pressing the platform down may move the platform from the elevated configuration to a depressed configuration, in which a distance between the platform and the base portion and/or other portion of the adapter may be less than in the elevated configuration.

At step 1006, the process 1000 involves latching the first medical instrument to secure the first medical instrument to the platform and/or to secure the platform in the depressed configuration. For example, latching the first medical instrument may prevent the platform from being raised to the elevated configuration. Latching the first medical instrument may involve use of one or more latches and/or latching mechanisms at the base portion and/or other portion of the adapter. When the first medical instrument and/or platform are pressed downwards (i.e., towards the base portion) attachment features (e.g., grooves, edges, indentations, latches, etc.) at the first medical instrument may be configured to latch to corresponding features at the adapter. For example, an edge portion of the first medical instrument may be configured to press one or more latching mechanisms inwards to overcome an outward bias of the latching mechanisms and/or one or more indentations at the first medical instrument may be configured to allow the latching mechanisms to return to a biased configuration to lock the first medical instrument in place.

At step 1008, the process 1000 involves attaching a second medical instrument at a second docking portion of the adapter to prevent release of the first medical instrument from the adapter. For example, the second medical instrument, when docked at the second docking portion of the adapter, may be configured to at least partially cover and/or otherwise prevent activation of a release mechanism at the adapter which may be configured to de-latch the first medical instrument from the adapter. In this way, the first medical instrument may be prevented from being removed when removing the first medical instrument can cause damage to the patient.

In some embodiments, the second medical instrument may be a working channel tool (e.g., a basketing tool) having at least a portion configured to fit into a receptor of the first medical instrument.

At step 1010, the process 1000 involves removing the second medical instrument from the second docking portion of the adapter. The second medical instrument may be attached and/or docked at the adapter for use in performing a medical procedure (e.g., grasping a kidney stone within a patient). When the medical procedure is completed, the second medical instrument may be removed from the adapter. In some embodiments, removing the second medical instrument may involve activation a release button and/or similar device at the second medical instrument and/or adapter to disengage latching mechanisms at the second medical instrument and/or adapter. Moreover, removing the second medical instrument may involve detaching the second medical instrument from the first medical instrument. For example, a sheath and/or medical extending from the second medical instrument may be removed from a receptor of the first medical instrument. In another example, a breakable attachment between the first medical instrument and the second medical instrument (e.g., using magnetic elements) may be broken to allow removal of the second medical instrument.

At step 1012, the process 1000 may involve activating a release mechanism at the adapter to release and/or allow removal of the first medical instrument from the adapter. The release mechanism may be a device configured to move along at least a portion of the adapter and/or may be configured to press against one or more torsion springs biasing latching mechanisms at the adapter in an outward configuration. The release mechanism may be configured to press the torsion springs inwards, thereby pressing one or more latching mechanisms inwards and/or dislodging the one or more latching mechanisms from indentations and/or other attachment features of the first medical instrument.

At step 1014, the process 1000 may involve removing the first medical instrument from the adapter and/or platform and/or allowing the platform to return to an elevated configuration.

### Multi-Interface Drive Outputs

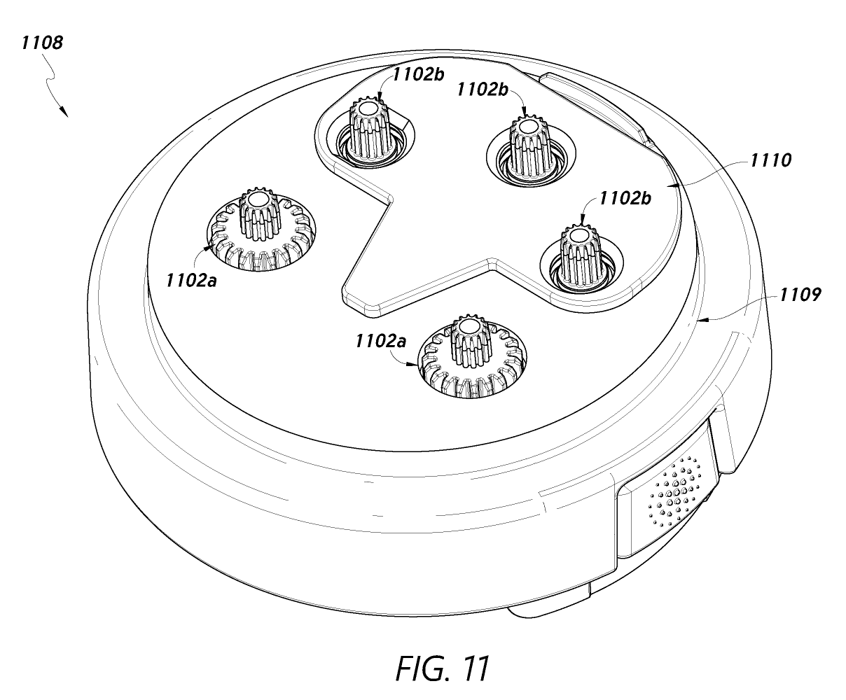

FIG. 11 illustrates at least a portion of a drive assembly including an adapter 1108 configured to drive multiple types of medical instruments, in accordance with one or more embodiments. The adapter 1108 may comprise any number of drive outputs 1102 and/or can include different types of drive outputs 1102. For example, a set of first drive outputs 1102a may be configured to drive at least a first type of medical instrument and/or a set of second drive outputs 1102b may be configured to drive a second type of medical instrument. Each drive output 1102 may be configured to extend from a base portion 1109 of the adapter 1108. While the adapter 1108 is shown including two first drive outputs 1102a and three second drive outputs 1102b, the adapter 1108 can include any number of drive outputs 1102 of either type of drive output. In some embodiments, the drive assembly may comprise multiple types of drive outputs. For example, the first drive outputs 1102a may be a first type of drive output and/or the second drive outputs 1102b may be a second type of drive output.

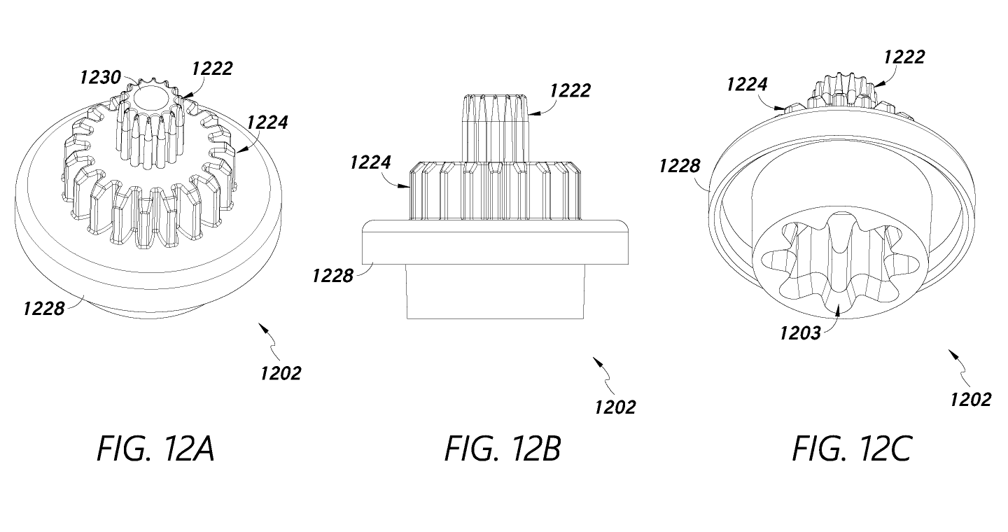

Moreover, some drive outputs (e.g., the first drive outputs 1102a) may be configured to drive multiple types of medical instruments. For example, a first type of drive output may include a first drive interface configured to drive a first type of medical instrument and/or a second drive interface configured to drive a second type of medical instrument (see, e.g., FIGS. 12A-12C).

One or more drive outputs 1102 may be associated with a first docking portion including a platform 1110, which may extend from the base portion 1109. For example, one or more drive outputs 1102 may be configured to pass at least partially through one or more apertures of the platform 1110. In some embodiments, the platform 1110 may be extendable between a raised and/or lowered configuration. While the second drive outputs 1102b are shown passing at least partially through the platform 1110, first drive outputs 1102a (which may be configured to drive multiple types of medical instruments) may be associated with and/or pass at least partially through the platform 1110 in some embodiments.

FIGS. 12A-12C illustrate a drive output 1202 configured to drive multiple types of medical instruments, in accordance with one or more embodiments. The drive outputs 1202 may comprise a first drive interface 1222 (e.g., a spline) configured to drive a first type of medical instrument (e.g., a catheter tool) and/or a second drive interface 1224 (e.g., a gear) configured to drive a second type of medical instrument (e.g., a basketing tool). In some embodiments, the first drive interface 1222 and the second drive interface 1224 may be coaxial and/or may be configured to rotate about a common axis. The first drive interface 1222 and/or the second drive interface 1224 may have a generally circular form (e.g., when viewed from above). In some embodiments, the first drive interface 1222 may have a first diameter that is less than a second diameter of the second drive interface 1224 and/or the second diameter is greater than the first diameter. The first drive interface 1222 and/or the second drive interface 1224 may comprise various engagement features (e.g., meshing gears) which may be configured to mesh and/or otherwise engage with corresponding drive inputs of one or more medical instruments.

In some embodiments, the first drive interface 1222 and the second drive interface 1224 may be configured in a tiered orientation in which one of the first drive interface 1222 and the second drive interface 1224 may extend a greater distance from a base portion 1228 of the drive output 1202. For example, the first drive interface 1222 may extend through and/or from the second drive interface 1224 and/or may extend a greater distance from the base portion 1228 than the second drive interface 1224. The first drive interface 1222 may comprise a distal tip 1230 which may be situated at a maximal distance of the drive output 1202 from the base portion 1228.

The first drive interface 1222 and/or the second drive interface 1224 may be configured to cause a gear ratio change from a drive output of a robotic arm to one or more medical instruments. For example, the first drive interface 1222 and/or second drive interface 1224 may be configured to translate a lower rotational speed at an adapter to a higher rotational speed at one or more medical instruments driven by the drive interfaces. A gear ratio change may enable relatively fast linear travel of opening/closing and/or inserting/retracting of the one or more medical instruments. In some embodiments, the first drive interface 1222 may be configured to drive a first medical instrument and/or a first type of medical instrument with a first drive ratio and/or the second drive interface 1224 may be configured to drive a second medical instrument and/or a second type of medical instrument with a second drive ratio that is different than the first drive ratio.

FIG. 12C illustrates an underside of the drive output 1202. In some embodiments, the drive output 1202 may comprise a drive input 1203 configured to receive one or more outputs from a robotic system (e.g., a robotic arm). A tiered and/or multi-interface drive assembly can enable the drive assembly to be interchangeably used for different patients and/or different portions of a robotic system.

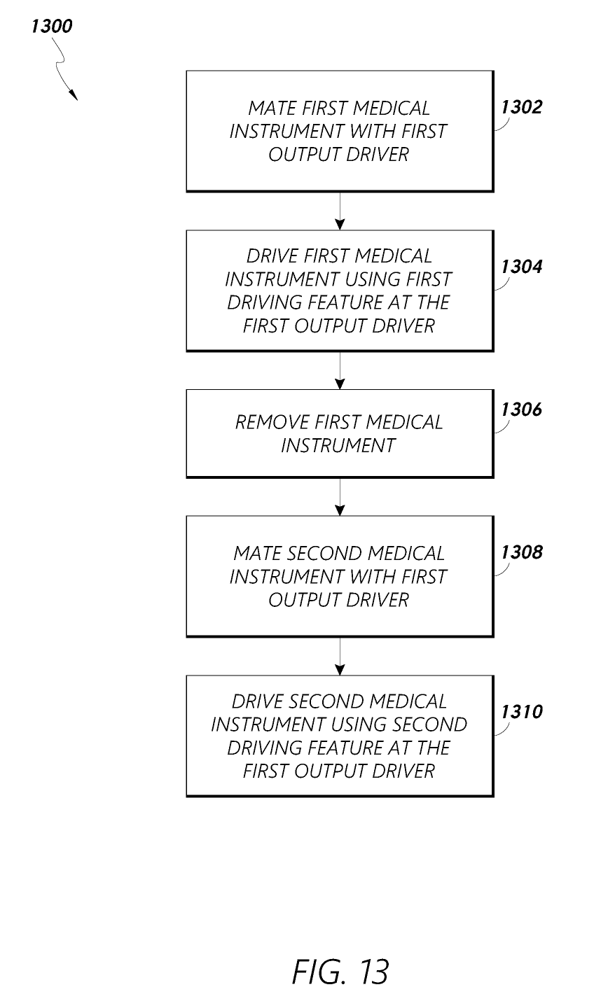

FIG. 13 provides a flow diagram for a process 1300 for driving one or more medical instruments at a drive assembly which may comprise one or more adapters (e.g., sterile adapters) for transferring force from a robotic system in accordance with one or more embodiments.

At step 1302, the process 1300 involves mating and/or attaching a first medical instrument with/to one or more output drivers (e.g., drive outputs) including a first output driver. The first output driver may comprise multiple driving features and/or interfaces (e.g., a first interface and/or a second interface) and/or each of the multiple interfaces may be configured to drive different types of medical instruments. The first medical instrument may be a first type of medical instrument configured to be driven using a first type of interface (e.g., the first interface). In some embodiments, mating the first medical instrument with the first output driver may involve at least partially covering the first output driver and/or the first interface with a corresponding drive input of the first medical instrument.

At step 1304, the process 1300 involves driving the first medical instrument using the first interface of the first output driver. In some embodiments, the first interface may comprise a network of meshing teeth and/or similar features configured to mesh with corresponding features at the first medical instrument.

At step 1306, the process 1300 involves removing the first medical instrument and/or disengaging the first medical instrument from the first interface and/or the first output driver. In some embodiments, removing the first medical instrument may involve activating a release mechanism at the adapter.

At step 1308, the process 1300 involves mating a second medical instrument with the first output driver following removal of the first medical instrument from the first output driver. The second medical instrument may be a second type of medical instrument configured to be driven using a second type of drive interface. The first output driver may comprise a second interface that is the second type of drive interface and/or is configured to drive the second medical instrument.

At step 1310, the process 1300 involves driving the second medical instrument using the second interface and/or driving feature at the first output driver. In some embodiments, the second interface may comprise a network of meshing teeth and/or similar features configured to mesh with corresponding features at the second medical instrument.

Described herein are systems, devices, and methods to facilitate interaction between one or more medical instruments and one or more medical instrument drive assemblies, in connection with certain medical procedures. In particular, systems, devices, and methods in accordance with one or more aspects of the present disclosure can facilitate guiding one or more medical instruments to a desired configuration at and/or with respect a medical instrument drive assembly, driving the one or more medical instruments, and/or managing removal of the one or more medical instruments from the medical instrument drive assembly. Interaction between one or more medical instruments and one or more medical instrument drive assemblies in accordance with the various embodiments disclosed herein can advantageously simplify and/or reduce certain risks associated with use of the one or more medical instruments.

Some implementations of the present disclosure relate to a medical instrument drive assembly comprising at least a base portion, a first set of one or more drive outputs coupled to and/or extending from the base portion and configured to drive a first medical instrument, and a platform secured to the base portion and movable between an elevated configuration and a depressed configuration. The platform is biased in the elevated configuration. The medical instrument drive assembly further comprises a latching mechanism configured to cause the platform to be held in the depressed configuration.

The medical instrument drive assembly may further comprise a second set of one or more drive outputs configured to drive a second medical instrument. The platform can be configured to guide the first medical instrument onto the first set of one or more drive outputs.

The platform can be configured to attach to the first medical instrument. In some embodiments, the medical instrument drive assembly further comprises a docking portion configured to receive a second medical instrument.

In some embodiments, the medical instrument drive assembly further comprises a release mechanism at the docking portion configured to disengage the latching mechanism to allow the platform to be moved to the elevated configuration. The second medical instrument, when docked at the docking portion, can prevent activation of the release mechanism and/or at least partially cover the release mechanism.

The platform may comprise one or more apertures configured to receive at least a first drive output of the first set of one or more drive outputs. The first drive output can be situated within a first aperture of the one or more apertures when the platform is in the depressed configuration. A first aperture of the one or more apertures can be suspended and/or situated over the first drive output when the platform is in the elevated configuration.

In some embodiments, the first medical instrument is a scope and/or endoscope device. The platform may comprise one or more magnetic elements configured to mate with one or more corresponding magnetic elements at the first medical instrument. The one or more magnetic elements of the platform may comprise a first magnetic element having a first polarity and a second magnetic element having a second polarity.

The medical instrument drive assembly may further comprise one or more springs situated below at least part of the base portion and/or between at least a portion of the base portion and at least a portion of the platform and configured to bias the platform in the elevated configuration. Pressing the platform to the depressed configuration can cause the latching mechanism to secure the platform in the depressed configuration and/or the first medical instrument to the platform.

In some embodiments, the latching mechanism can be configured to cause the platform to be held in the depressed configuration by securing the first medical instrument to the platform. The base portion may comprise one or more drive inputs for interacting with driving features of one or more robotic arms.

In some implementations, the present disclosure relates to a medical instrument drive assembly comprising a first docking portion configured to receive a first medical instrument, a first set of one or more drive outputs associated with the first docking portion and configured to drive the first medical instrument, a latch configured to secure the first medical instrument to the first docking portion, a release configured to disengage the latch to allow the first medical instrument to be displaced from the first docking portion, and a second docking portion configured to receive a second medical instrument in a manner in which the second medical instrument prevents activation of the release.

The second medical instrument may comprise one or more attachment mechanisms configured to attach to the release. The second medical instrument can be a working channel tool configured to fit into a working channel of the first medical instrument. The second medical instrument may comprise one or more alignment features configured to mate with corresponding alignment features of the first medical instrument.

Some implementations of the present disclosure relate to a medical instrument drive assembly comprising a first docking portion configured to receive a first medical instrument, a first set of one or more drive outputs coupled to and/or extending from the first docking portion and configured to drive the first medical instrument, and one or more magnetic elements associated with the first docking portion and configured to guide alignment of the first set of one or more drive outputs with one or more drive inputs of the first medical instrument.

A first magnetic element of the one or more magnetic elements can have a first magnetic polarity and a second magnetic element of the one or more magnetic elements can have a second magnetic polarity. The one or more magnetic elements can be configured to mate with corresponding magnetic elements at the first medical instrument.

In some embodiments, the medical instrument drive assembly further comprises a platform extending from the first docking portion that is movable between an elevated configuration and a depressed configuration. The one or more magnetic elements can be attached to the platform.

In some implementations, the present disclosure relates to a medical instrument drive assembly comprising a base portion and one or more drive outputs coupled to and/or extending from the base portion and configured to drive a first medical instrument. At least a first drive output of the one or more drive outputs includes a first drive interface configured to drive a first type of medical instruments and a second drive interface configured to drive a second type of medical instruments.

The first drive output of the one or more drive outputs comprises mesh engagement features configured to mesh with a corresponding drive input of the first medical instrument. In some embodiments, the first drive interface and the second drive interface are coaxial.

In some embodiments, a diameter of the second drive interface is greater than a diameter of the first drive interface. The first drive interface can be configured to drive the first type of medical instrument with a first drive ratio. The second drive interface can be configured to drive the second type of medical instrument with a second drive ratio that is different than the first drive ratio.

For purposes of summarizing the disclosure, certain aspects, advantages and novel features have been described. It is to be understood that not necessarily all such advantages may be achieved in accordance with any particular embodiment. Thus, the disclosed embodiments may be carried out in a manner that achieves or optimizes one advantage or group of advantages as taught herein without necessarily achieving other advantages as may be taught or suggested herein.

### Additional Embodiments

Depending on the embodiment, certain acts, events, or functions of any of the processes or algorithms described herein can be performed in a different sequence, may be added, merged, or left out altogether. Thus, in certain embodiments, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain embodiments require at least one of X, at least one of Y and at least one of Z to each be present.

It should be appreciated that in the above description of embodiments, various features are sometimes grouped together in a single embodiment, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular embodiment herein can be applied to or used with any other embodiment(s). Further, no component, feature, step, or group of components, features, or steps are necessary or indispensable for each embodiment. Thus, it is intended that the scope of the inventions herein disclosed and claimed below should not be limited by the particular embodiments described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example embodiments belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. A medical instrument drive assembly, comprising:
a base portion; and
one or more drive outputs rotatably coupled to the base portion, at least one of the one or more drive outputs including:
a first drive interface configured to mate with and drive a first type of medical instrument; and
a second drive interface configured to mate with and drive a second type of medical instrument.

2. The medical instrument drive assembly of claim 1, wherein the at least one of the one or more drive outputs comprises a drive input configured to mate with an output of a robotic system.

3. The medical instrument drive assembly of claim 1, wherein the first drive interface and the second drive interface are coaxial.

4. The medical instrument drive assembly of claim 1, wherein an outer diameter of the second drive interface is greater than an outer diameter of the first drive interface.

5. The medical instrument drive assembly of claim 1, wherein the first drive interface is configured to drive the first type of medical instrument with a first drive ratio and the second drive interface is configured to drive the second type of medical instrument with a second drive ratio that is different than the first drive ratio.

6. The medical instrument drive assembly of claim 1, wherein the first drive interface comprises mesh engagement features sized to mesh with a corresponding first drive input of the first type of medical instrument and the second drive interface comprises mesh engagement features sized to mesh with a corresponding second drive input of the second type of medical instrument.

7. The medical instrument drive assembly of claim 1, wherein the at least one of the one or more drive outputs comprises a first drive output, and wherein the one or more drive outputs further comprises a second drive output.

8. The medical instrument drive assembly of claim 7, further comprising:
a platform secured to the base portion and movable between an elevated configuration and a depressed configuration, the platform being biased to the elevated configuration, wherein:
in the depressed configuration, at least the second drive output extends through and beyond the platform, and
in the elevated configuration, the platform extends beyond at least a portion of the second drive output.

9. The medical instrument drive assembly of claim 8, further comprising a latching mechanism configured to cause the platform to be held in the depressed configuration.

10. The medical instrument drive assembly of claim 1, wherein the at least one of the one or more drive outputs includes a base disposed within the base portion, and wherein the first drive interface extends from the base a first distance, and the second drive interface extends from the base a second distance greater than the first distance.

11. The medical instrument drive assembly of claim 1, wherein the first drive interface extends from the second drive interface and provides a distal tip situated at a maximal distance of the at least one of the one or more drive outputs from the base portion.

12. A medical instrument drive assembly, comprising:
a base portion; and
a first drive output coupled to the base portion and rotatable relative to the base portion about a first axis, the first drive output including:
a base disposed in the base portion;
a first drive interface extending from the base and having a first outer diameter;
a second drive interface extending from the first drive interface and having a second outer diameter.

13. The medical instrument drive assembly of claim 12, wherein the first outer diameter is greater than the second outer diameter.

14. The medical instrument drive assembly of claim 13, wherein the first drive interface and the second drive interface are coaxial.

15. The medical instrument drive assembly of claim 12, further comprising a second drive output coupled to the base portion and rotatable about a second axis.

16. The medical instrument drive assembly of claim 15, further comprising a platform secured to the base portion and movable between an elevated configuration and a depressed configuration, the platform being biased to the elevated configuration, wherein:
in the depressed configuration, at least the second drive output extends through and beyond the platform, and
in the elevated configuration, the platform extends beyond at least a portion of the second drive output.

17. The medical instrument drive assembly of claim 16, further comprising a latching mechanism configured to cause the platform to be held in the depressed configuration.

18. A method, comprising:
engaging a first drive input of a first medical instrument with a first drive interface of a drive output of a drive assembly;
operating the first medical instrument in response to driving the first drive input with the first drive interface of the drive output;
disengaging the first drive input from the drive output;
engaging a second drive input of a second medical instrument with a second drive interface of the drive output; and
operating the second medical instrument in response to driving the second drive input with the second drive interface of the drive output.

19. The method of claim 18, wherein the first drive interface has an outer diameter greater than the outer diameter of the second drive interface.

20. The method of claim 18, wherein disengaging the first drive input includes activating a release mechanism of the drive assembly.
